(19) United States
(12) Patent Application Publication
(10) Pub. No.: US 2018/0256633 A1
(43) Pub. Date: Sep. 13, 2018

(54) LONG CHAIN INULIN FOR STIMULATING AN IMMUNE RESPONSE

(71) Applicants: Rijksuniversiteit Groningen, Groningen (NL); Academisch Ziekenhuis Groningen, Groningen (NL); Koninklijke Coöperatie Cosun U.A., Breda (NL)

(72) Inventors: Paulus DE VOS, Groningen (NL); Elizabeth Elaine VAUGHAN, Breda (NL); Leonie Marloes VOGT, Groningen (NL)

(21) Appl. No.: 15/760,539

(22) PCT Filed: Sep. 19, 2016
(86) PCT No.: PCT/EP2016/072103

§ 371 Date: Mar. 15, 2018

(30) Foreign Application Priority Data
Sep. 18, 2015 (EP) 15185963.4

(51) Int. Cl. A61K 31/733; A61K 39/29; A61K 39/39; A61P 37/02; A23L 33/125

(52) U.S. Cl. CPC A61K 31/733; A61K 39/29; A61K 2039/55583; A61P 37/02; A23L 33/125; A61K 39/39

(57) ABSTRACT

The invention relates to a long chain inulin for influencing the immune response against a pathogen. The invention also relates to a combination comprising a long chain inulin and a vaccine for influencing the immune response against a pathogen, wherein the long chain inulin is orally administrated.

### FIELD OF THE INVENTION

The invention relates to a combination of a long chain inulin and a vaccine, wherein the long chain inulin is orally administered. The invention also relates to a long chain inulin for influencing the immune response against a pathogen wherein the long chain inulin is orally administered.

### BACKGROUND OF THE INVENTION

Dietary fibers are considered an essential part of healthy nutrition. The health benefits of sufficient fiber intake comprise prevention of colorectal cancer¹, type 2 diabetes², cardiovascular disease³, reduced risk of hyperlipidemia, hypercholesterolemia and hyperglycemia^{4 5}, and regulation of bowel habit⁶⁻¹⁰.

Inulin-type fructans are prebiotic dietary fibers with many health benefits. They are oligomers and polymers of fructose subunits, and often terminate in a glucose molecule¹³. Besides the established effects on gut health and metabolism^{11 12 14}, evidence for immunostimulatory effects of inulin-type fructan consumption is accumulating. Results from ex vivo and in vitro experiments in human cells, cell lines, and from animal studies, support the notion that inulin-type fructans exert immune modulating effects¹⁵⁻¹⁹. The underlying mechanisms for immune modulation by inulin-type fructans have been attributed to the selective stimulation of beneficial bacteria in the intestine, and their SCFA fermentation products¹⁷. In addition, previous results from our group support the notion that these fibers can also exert direct effects on immune cells, by activating or inhibiting Toll-like receptors (TLRs) on monocytic cells, and modulating, preferably inducing cytokine production in human peripheral blood mononuclear cells (PBMCs) upon in vitro stimulation²⁰.

There is no evidence that inulin-type fructan supplementation could behave as a compound that is able to quantitatively and qualitatively influence the immune response induced in response to a pathogen or to an antigen from a pathogen such as present in a vaccine in humans. Results obtained so far with infants to elderly from supplementation trials²¹⁻²⁴ are not consistent for at least the following reasons. The compound used as “inulin-type fructan” is not always well defined in terms of degree of polymerization (DP), and often mixtures of fructans having different DP and/or with other carbohydrates are applied. This study demonstrates that the effects on the immune system is at least depending on the DP of the inulin-type fructan used. In addition, several studies treated infant or toddler populations or use different types of infant/toddler populations within one single study. The immune system of an infant population is expected to be Th2 and less susceptible to a skewing towards Th1 than the one of an older infant/toddler population, let alone the one of an elderly population. Absence of significant effects observed in one specific population does not per se indicate the same absence of significant results would be obtained in another population having an immune system more susceptible to be skewed towards a Th1 immune response.

There is always a need for compounds that could further optimize the efficacy of vaccines or promote immunity against infections. Ideally such compounds may also confer new attractive properties to vaccines. For example, they can influence the type and direction of the immune response induced.

### DESCRIPTION OF THE INVENTION

We surprisingly show that long chain inulin-type fructan supplementation influences the immunity response against pathogens such as during vaccination, where an immune reaction is evoked against a pathogen without inducing a disease. Using common vaccination programs against hepatitis B surface antigen (HBsAg) to immunize subjects, we demonstrate that by supplementing young adults with inulin-type fructans from 7 days before, until 7 days after the first injection of an HBsAg vaccination program, the antibody response against the vaccine has been significantly improved compared to placebo supplemented participants. This has been shown by an earlier onset of titer development, an increased titer response on the final measuring time point, and/or the presence and number of ‘responders’, characterized as subjects with titers equal to or above 10 IU/mL. More importantly, we demonstrated that in the treated group, a shifted Th1/Th2 balance may have been induced which was skewed towards Th1 cell responses. This is the first in vivo study which demonstrates a structure-function relation of a dietary fiber on a human immune response.

### Combination

In a first aspect, the invention relates to a combination comprising a long chain inulin and a vaccine, wherein the long chain inulin is orally administered. In said first aspect, the combination is preferably for influencing the immune response against a pathogen. In a second aspect, the invention also relates to a long chain inulin for influencing the immune response against a pathogen, wherein the long chain inulin is orally administered.

Unless otherwise indicated, the definition and text of the description applies to both aspects.

In the first aspect of the invention, the word “combination” preferably means that a long chain inulin and a vaccine are not comprised within one single composition. The invention provides the insight that both compounds (i.e. a long chain inulin and a vaccine) are needed or are used in order to get an optimal or maximum or significant or measurable effect on the immune response as defined later on. If a long chain inulin and a vaccine are not present in a same composition, each compound may be used sequentially or simultaneously administered.

However it is not excluded that a long chain inulin and a vaccine may be together or present together or combined together or physically in contact with the other forming one single composition.

Inulin is the generic name covering all β(2,1) fructans (see FIG. 1). Inulin is a fructan polymer or fructan oligomer. It comprises chain-terminating glucosyl moieties and a repetitive fructosyl moiety, which are linked by β(2,1) bonds. Inulin as used herein refers not only to poly-β-D-(2→1)-fructofuranosyl-α-D-glucopyranose but also to derivatives thereof such as poly-β-D-(2→1) polyfructofuranose. The latter may be obtained by hydrolysis, by chemical or by enzymatic means, for example by removal of the end glucose from inulin, for example using an invertase or inulase enzyme capable of removing the end glucose. The term inulin also refers to any natural or synthetic inulin for example fructooligosaccharides synthesised from sucrose.

Long chain inulin refers to the degree of polymerisation of inulin. The degree of polymerization (DP) or number of fructose units in the fructose polymer of standard/native inulin ranges from mainly 2 to 60 or from 2 to 60 with average DP of approximately 8-13 or 8-13 for example for native chicory. Long chain inulin is generally obtained by crystallization and removal of impurities. Long chain inulin therefore is a fructose polymer made of poly-β-D-(2→1)-fructofuranosyl-α-D-glucopyranose but also of derivatives thereof such as poly-β-D-(2→1) polyfructofuranose and which has preferably a DP ranged from mainly 8 to 60 or from 8 to 60. In this context, mainly preferably means that at least 60%, 70%, 80%, 90%, 95% or 99% of the molecules has its DP comprised in this range. It is also encompassed by the invention that long chain inulin has a DP ranged from mainly 10-60, 14-30, 10-42 or mainly 20-30, 21-30, or mainly 21-29 of from 10-60, 10-42, 14-30, 20-30, 21-30, 21-29. A preferred average DP of long chain inulin is 20, 21, 22, 23, 24, 25, 26, 27, 28, 29, 30.

Short chain inulin made from standard inulin ranges from 2 to 35 with an average DP of ranged from mainly 6-10 or 7-9 or from 6-10 or 7-9. Oligofructose and fructo-oligosaccharide (FOS) may also be considered as short chain inulin. Typically oligofructose has a DP of 2-8. Oligofructose may be produced from inulin by partial enzymatic hydrolysis. Typically FOS has a DP of 2-5 and may be enzymatically produced from sucrose.

An estimation of longest inulin chains present in for example chicory inulin can be made from an inulin chain length distribution analysed for example by a chromatographic method (HPAEC-PAD).

A preferred natural inulin is chicory (Cichorium intybus) inulin. Other sources for inulin production include artichoke, agave, amongst others. Inulin from chicory is a polydisperse mixture of linear fructan oligomers and polymers coupled by means of β(2-1) bonds, and mostly with a terminal glucose unit. Native inulin and its partial enzymatic hydrolysis product oligofructose (OF) are usually purified from the chicory plant (preferably from the root of the chicory plant) and its partial enzymatic hydrolysis product oligofructose (OF), and long-chain inulin, are produced (or are usually produced) from native inulin.

A preferred long chain inulin is Frutafit®TEX! from Sensus, Roosendaal, the Netherlands. Frutafit®TEX! is a natural powdered food ingredient based on or derived from native chicory inulin, and known for its texturizing properties. In order to obtain Frutafit®TEX!, chicory roots are first pretreated and subsequently the obtained mixture is purified. The pretreatment may comprise a washing, a slicing and extraction steps. The extraction step is preferably carried out using only hot water. Hot water extraction of inulin from chicory takes place at similar conditions of extraction of sugar from sugar beets. It means that preferably no other solvent is being used. Subsequently long chain inulin is generally obtained by crystallization and removal of impurities. (Meyer & Blaauwhood, 2009, edited by G. O. Philips and P. A. Williams, pages 829-848, Woodhead, Cambridge, UK).

The DP ranges of the Frutafit®TEX! product are from mainly 8 to 60 or from 8 to 60. In this context, mainly preferably means that at least 60%, 70%, 80%, 90%, 95% or 99% of the molecules of the product has its DP comprised in this range. More preferably, the DP of the product is ranged from mainly 14-30 or 14-30, 10-42, or mainly 20-30, 21-30, or mainly 21-29 or 20-30, 21-30 or 21-29. A preferred average DP of this product is 22, 23, 24, 25, 26, 27, 28, 29. Experimental evidence are provided in this application using a combination of the invention comprising Frutafit®TEX! as long chain inulin.

Suitable inulin derivatives included within the scope of this term are derivatives of inulin in which the free hydroxyl groups or parts thereof have been for example oxidised, acetylated, methylated, etherified or esterified, for example by chemical substitution with alkyl, aryl or acyl groups, by known methods or by enzymatic substitution.

The invention also encompasses a particle that is constituted by, contains or is coated by long chain inulin, or a derivative or mimetic thereof. The long chain inulin particle may be solid or hollow and may be wholly comprised of long chain inulin molecules or may alternatively have a non-sugar core, skeleton or shell comprising, for example, carbohydrate compounds, metal compounds, proteins or lipids but which at its surface expresses long chain inulin molecules either covalently or non-covalently bonded to the components comprising the core.

In one embodiment, long chain inulin as used in the present invention is at least partly soluble and/or is not detectable as a crystalline form at a temperature ranged from room temperature and body temperature of the subject to whom it is to be administered. Preferably, the solubility and absence of crystalline form of said long chain inulin is assessed when said long chain inulin is present at a concentration of no greater than 0.5 mg/ml or 1 mg/ml or 2 mg/ml in distilled water or saline or phosphate buffered saline, for at least 10, 20, 30, 40, 50, or 60 minutes.

Therefore a preferred combination of the first aspect of the invention comprises a long chain inulin with a DP of mainly 10-60, (or a DP of 10-60), preferably an average DP of 22, 23, 24 and a vaccine, wherein the long chain inulin is orally administered.

Accordingly in the second aspect of the invention, a long chain inulin with a DP of mainly 10-60, (or a DP of 10-60), preferably an average DP of 22, 23, 24 is orally administered for influencing the immune response against a pathogen.

The dose of long chain inulin used in the invention may vary depending from the subject treated. The subject treated is an animal, more preferably a mammal, even more preferably a human. In a further preferred embodiment, a human subject is a baby (0 to 1 year), an infant or a toddler/small child (1 year to less than 5 years), a child (5 to 18 years) an adult (18-60 years) or an elderly (60 years and older). Other preferred animals include companion animal as dog, cat. Other preferred animals include pig, cows, horse.

In an embodiment, the dose of long chain inulin is ranged from 1 to 25 g per day for human adult and elderly, or 1 to 8 g per day or 1 to 5 g per day.

In another embodiment, the dose of long chain inulin is ranged from 1 to 15 g per day for children and small children, or 1 to 10 g per day or 1 to 8 g per day, or 1 to 5 g per day. The dose may be further adapted depending on the age of the children.

In another embodiment, the dose of long chain inulin is ranged from 0.01 to 15 g per day for baby and toddler/small child, or 0.1 to 12 g per day or 0.5 to 8 g per day, or 1 to 5 g per day. The dose may be further adapted depending on the age of the baby. Therefore in one embodiment, the dose of long chain inulin is ranged from:
1 to 25 g per day for human adult, or 1 to 8 g per day or 1 to 5 g per day,
1 to 15 g per day for infant or small children, or 1 to 10 g per day or 1 to 8 g per day, or 1 to 5 g per day or
0.01 to 15 g per day for baby, or 0.1 to 12 g per day or 0.5 to 8 g per day, or 1 to 5 g per day.

It is therefore clear to a skilled person that the long chain inulin may be for use in baby, small children or infant, adult or elderly. Preferably, the combination is for small children to elderly.

In a combination of the invention (first aspect), a vaccine is present. A vaccine or vaccine composition is a composition that provides active acquired immunity to a particular disease. A vaccine usually comprises an agent that resembles a disease-causing micro-organism or disease-causing virus. A disease-causing microorganism (or microorganism) or disease-causing virus (or virus) is also called a pathogen. A preferred disease-causing microorganism or a preferred disease-causing virus is such that it is able to induce a systemic infection. In a preferred embodiment, a vaccine comprises an antigen from said microorganism or virus. It is clear for the skilled person that in a preferred embodiment, a vaccine does not comprise or consist of an alive and/or whole pathogen. Usually if a pathogen is used in a vaccine it is killed or attenuated.

As used herein, the term “antigen” generally refers to a substance that evokes an immune response, including humoral immunity and/or cellular immunity response, and that is capable of binding with a product, e.g., an antibody or a T cell, of the immune response. An antigen as intended herein may in an alternative be such as to induce immuno-tolerance, e.g., may be an auto-antigen (including auto- and allo-antigens) or may be allergen. Hence, in a preferred example, an antigen requires a functioning immune system of a subject to which it is administered to elicit a physiological response from such a subject. The “antigen” as intended herein also encompasses “self-antigens” which do not provoke an immune response in a healthy individual but would do so in a person suffering from auto-immune disease, i.e. the failure of an organism to recognize its own constituent parts (down to the sub-molecular levels) as “self”, which results in an immune response against its own cells and tissues. Any disease that results from such an aberrant immune response is termed an autoimmune disease.

Accordingly, the “antigen” as intended herein also encompasses a (physiologically active) protein which would not provoke an immune response in a healthy individual but would do so in a person genetically deficient in said protein. In addition, the “antigen” as intended herein also encompasses an allergen which would not provoke an immune response in a healthy individual but would do so in a person suffering from an allergic disease.

An antigen as intended herein may be derived from any polypeptide to which an immune response in a human or animal subject would be therapeutically useful, e.g., from a pathogen, e.g., from a viral, prokaryotic (e.g., bacterial) or eukaryotic pathogen, from a non-physiological protein (e.g., a protein derived from cancer tissue), from allergen (e.g., for eliciting immune tolerance), etc. An antigen could also be a metabolite of a protein. As an example, the antigen could be a polysaccharide, a lipid or other.

An antigen which is able to induce a specific immune response from a subject when said antigen is present in said subject is said to be immunogenic or to be an immunogen. An antigen is preferably a polypeptide or a peptide. A peptide may comprise 6, 7, 8, 9, 10, 11, 12, 13, 14, 15, 16, 17, 18, 19, 20, 21, 22, 23, 24, 25, 26, 27, 28, 29, 30 amino acids or more. An antigen may be a protein fragment or a full length protein originating from a disease-causing organism such as a microorganism or virus as identified later herein. It is also possible to use several antigens from the same organism in order to be more effective in combating the organism. An antigen may also be defined by reference to an encoding nucleic acid molecule represented by a nucleic acid sequence.

The source of an antigen may be a protein, a digest of the protein and/or a fragment thereof, which may be in a purified form or may be comprised within a crude composition, preferably of biological origin, such as a bacterial lysate, parasitic lysate, yeast lysate, viral lysate, fungal lysate, sonicate or fixate. Alternatively, an antigen may be chemically synthesized or enzymatically produced in vitro. The source of a protein, or fragment thereof as antigen, may also be a nucleic acid encoding said, or fragment thereof, from an RNA or DNA template. The RNA or DNA molecules may be ‘naked’ DNA, preferably comprised in vesicles or liposomes, or they may be comprised in a nucleic acid construct or a vector. The vector may be any (recombinant) DNA or RNA vector known in the art, and preferably is a plasmid; wherein genes encoding latency antigens are operably linked to regulatory sequences conferring expression and translation of the encoded messengers. The vector may also be any DNA or RNA virus, such as, but not limited to, Adenovirus, Adeno-Associated Virus (AAV), a retrovirus, a lentivirus, modified Vaccinia Ankara virus (MVA) or Fowl Pox virus, or any other viral vector capable of conferring expression of a polypeptide into a chosen subject. DNA vectors may be non-integrating, such as episomally replicating vectors, or may be vectors integrating in the host genome by random integration or by homologous recombination.

DNA molecules comprising genes encoding an antigen protein, or fragments thereof according to the current invention, optionally embedded in a vector such as a virus or plasmid, may be integrated in a genome of a subject. In a preferred embodiment of the invention, such a host may be a micro-organism. Preferably such a recombinant micro-organism is a Mycobacterium, for instance of the species M. tuberculosis, M. smegmatis or M. bovis and most preferably M. bovis Bacillus Calmette Guerin (BCG) or M. smegmatis, capable of delivering to a host the polypeptides or fragments thereof according to the invention (as described in Yue. Y. et al, (2007), J. Virol. Meth., 141: 41-48, Cayabiyab Y. et al, (2006), J. Virol., 80: 1645-1652). Recombinant BCG and methods for recombination are known in the art; for instance, in WO2004094469.

An antigen may originate from any organism known to be associated with a disease or a condition in a subject. An antigen may originate from a microorganism such as a bacterium, a yeast, a fungus, a parasite. Alternatively, an antigen may originate from a virus. An antigen may also be a self or auto antigen as, for example, those associated or linked to allergic diseases.

In an embodiment, a vaccine as used in a combination of the invention comprises an antigen and said antigen is from a virus. Any virus that causes a disease in humans from which antigens are known is encompassed within the scope of the present invention. Such virus include an Adenovirus, a Coxsackievirus, an Epstein Barr virus (EBV), a Hepatitis A, B or C virus, a Herpes simplex virus type I or II or VIII, a cytomegalovirus, a HIV virus, an influenza virus, a measles virus, a mumps virus, a human papilloma virus, a parainfluenza virus, a polio virus, a rabbies virus, a respiratory syncytial virus (RSV), a rubella virus, a Varicella zoster-virus and an Ebola virus.

An adenovirus may cause acute febrile pharyngitis, pharyngoconjunctival fever, epidemic keratoconjunctivitis and/or infantile gastroenteritis.

A coxsackievirus may cause hand, foot and mouth disease, aseptic meningitis, pericarditis and/or myocarditis.

An EBV may cause infectious mononucleosis, Burkitt's lymphoma, Hodgkin's lymphoma and/or nasopharyngeal carcinoma.

A hepatitis A virus may cause an acute hepatitis.

A hepatitis B or C virus may cause acute hepatitis, chronic hepatitis, hepatic cirrhosis and/or hepatocellular carcinoma. Experimental evidence are provided in this application using a combination of the invention comprising a hepatitis B virus.

A Herpes simplex virus type I or II may cause a primary or latent infection. A herpes simplex type II may cause an aseptic meningitis.

A cytomegalovirus may cause an infectious mononucleosis and/or a Cytomegalic inclusion disease.

A HIV virus may cause AIDS.

An influenza virus may cause influenza, a measles virus may cause measles, a mumps virus may cause mumps.

A measles virus may cause measles and/or post-infectious encephalomyelitis.

A mumps virus may cause mumps.

A human papilloma virus may cause hyperplastic epithelial lesions, cervical carcinoma, and/or squamous cell carcinomas.

A parainfluenza virus may cause croup, pneumonia, bronchiolitis and/or common cold

A polio virus may cause poliomyelitis.

A rabies virus may cause rabies.

A RSV may cause bronchiolitis, pneumonia, influenza-like syndrome and/or severe bronchiolitis with pneumonia.

A rubella virus may cause German measles and/or congenital rubella.

A Varicella zoster-virus may cause chickenpox and/or herpes zoster.

An Ebola virus may cause the Ebola virus disease formerly known as Ebola haemorrhagic fever.

Therefore each of the listed virus infections and corresponding viral diseases may be treated using a combination of the invention.

In another embodiment, an antigen may originate from a yeast, a fungus, a bacterium or any other pathological cell. Any yeast or fungus or bacterium that causes a disease in humans from which antigens are known is encompassed within the scope of the present invention. Such bacteria include Mycobacterium tuberculosis, Streptococcus, Pseudomonas, Shigella, Campyobacter, Salmonella.

A Mycobacterium tuberculosis may cause tuberculosis.

A Streptococcus or Pseudomonas bacterium may cause pneumonia.

A Shigella, Campylobacter and Salmonella bacteria may cause a food-borne disease.

In another embodiment, an antigen may be an allergen.

In a preferred embodiment, the combination of the first aspect of the invention comprises a long chain inulin and a vaccine comprising an antigen from a pathogen, more preferably wherein the pathogen is a virus or a bacterium.

More preferably, the pathogen is able to induce a systemic viral or bacterial infection. Preferred bacteria inducing a systemic infection include: Mycobacterium tuberculosis, Streptococcus or Pseudomonas bacterium.

Preferred virus inducing a systemic infection include: Adenovirus, a Coxsackievirus, an Epstein Barr virus (EBV), a Hepatitis A, B or C virus, a Herpes simplex virus type I or II or VIII, a cytomegalovirus, a HIV virus, an influenza virus, a measles virus, a mumps virus, a human papilloma virus, a parainfluenza virus, a polio virus, a rabies virus, a respiratory syncytial virus (RSV), a rubella virus, a Varicella zoster-virus and an Ebola virus.

A most preferred virus is the hepatitis B virus.

In a second aspect of the invention, the oral administration of long chain inulin as defined herein is for influencing the immune response against a pathogen, preferably influencing the immune response against an antigen of a pathogen. In this aspect, a vaccine does not per se need to be administered. A subject may become infected or may become naturally infected by a pathogen. A pathogen and an antigen from a pathogen are as earlier defined herein. In this embodiment, preferred pathogens are those causing common infections like the influenza virus.

In a preferred embodiment of the first aspect, the combination comprises a long chain inulin and a vaccine, wherein the long chain inulin is orally administered to a human, the combination is for influencing the immune response of said human against a pathogen, preferably wherein the pathogen is a virus.

In a preferred embodiment of the second aspect, the long chain inulin is for influencing the immune response of a human against a pathogen, wherein the long chain inulin is orally administered and preferably wherein the pathogen is a virus.

The present invention demonstrates that oral administration of long chain inulin as defined herein (first and second aspects) influences the immune response against an antigen preferably present in a vaccine. In a more preferred embodiment, the immune response is systemic. Within the context of the invention, orally administered long chain inulin is said to influence the immune response elicited against an antigen preferably present in a vaccine when the elicited immune response has been changed, modified compared to the elicited immune response in the absence of the long chain inulin. The change in the immune response may be quantitative and/or qualitative. The change in the immune response is preferably assessed by comparison with the immune response in a control subject. To analyze the antigen-specific elicited immune response, said immune response is compared to the immune response induced in presence of an antigen preferably in the form of a vaccine without orally administered long chain inulin. An alternative or additional control may be a subject treated with an antigen, preferably in the form of the same vaccine and with a short chain inulin as defined herein. The induction is preferably assessed in a subject or in a sample from said subject or in cells from said subject. Preferred sample is blood and preferred cells are PBMCs.

In this context, the antigen-specific elicited immune response is synonymous with the induced immune response against said antigen or the increase in the induction of an immune response against said antigen or a detectable immune response against said antigen. Eliciting an antigen-specific immune response may be replaced with inducing, enhancing, or increasing an immune response against an antigen.

An immune response against said antigen may be a B and/or a T cell response. An immune response may be a B cell response, i.e. production of an antibody specifically directed against said antigen. An immune response may be a T cell response, preferably a Th1 response. The skilled person knows that depending on the disease, a B and/or T cell response may be required to be induced to control it. The production of said antibody could be assessed by ELISA, or by FACS preferably as carried out in the examples. Alternatively said immune response may be detected by measuring the production of cytokines such as, for example, IFNgamma, IL-6, TNFalpha, IL-12 or IL-10. The production of such cytokines could be assessed on PBMC cells of a treated subject by ELISA, PCR, or by a multiplex immunoassay built with luminex xmap technology from Biorad. Preferably the assay used for assessing the production of cytokines is the one used in the examples.

In short, in the context of the invention oral administration of long chain inulin as defined herein influences the immune response against an antigen preferably present in a vaccine in at least one of the following ways:
Cytokine production,
Monocyte/macrophage response and/or
B cell response and/or
T cell response.

The effects observed on cytokine production are preferably that:
the induction of TNFalpha, IL-12 and/or IFNgamma is higher with long chain inulin than with shorter chain inulin and/or
the production of IL-6 and/or IL-10 is lower with long chain inulin than with shorter chain inulin.

In the context of the invention, “higher” may be replaced by an increase of at least 10%, 15%, 20%, 25%, 30%, 35%, 40%, 45%, 50%, 55%, 60%, 65%, 70%, 75%, 80%, 85%, 90%, 95%, 100%, 150%, 200% or more.

In the context of the invention, “lower” may be replaced by a decrease of at least 10%, 15%, 20%, 25%, 30%, 35%, 40%, 45%, 50%, 55%, 60%, 65%, 70%, 75%, 80%, 85%, 90%, 95%, 100%, 150%, 200% or more.

The production of such cytokines may be assessed on a subject, or in a sample from said subject such as cells, preferably PBMC cells. The technique used for the assessment of the production of such cytokines is ELISA or PCR. The detection of the production of such cytokine may be carried out as in the examples. In an embodiment, the production of such cytokines is assessed after at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 day of administration of the combination of the invention (i.e. first aspect, vaccine and long chain inulin) or of administration of an antigen and the long chain inulin (second aspect).

The effects observed on B cell response are preferably that:
the antibody titer is increased by comparison to the antibody titer in a subject having only received the antigen preferably in a vaccine or the antigen preferably in a vaccine and short chain inulin and/or
the number of responders in a study is increased by comparison to the number of responders in a study having only received the antigen preferably in a vaccine or the antigen preferably in a vaccine and short chain inulin.

In the context of the invention, the word “increased” may mean an increase of at least 1%, 2%, 5%, 7%, 9%, 10%, 15%, 20%, 25%, 30%, 35%, 40%, 45%, 50%, 55%, 60%, 65%, 70%, 75%, 80%, 85%, 90%, 95%, 100%, 150%, 200% or more.

In the context of the invention, the number of responders is increased of at least 30%, 50%, 75%, 100%, 150%, 200%, 250% or more.

The assessment of antibody titer may be carried out on a subject, or in a sample from said subject such as in peripheral blood plasma samples. The technique used for the assessment of such titer may be an immunoassay. The assessment of such titer may be carried out as in the examples. In an embodiment, the assessment of such antibody titer is carried out after at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 day of administration of the combination of the invention (i.e. first aspect, vaccine and long chain inulin) or of administration of an antigen and the long chain inulin (second aspect). The number of responders is defined as the number of subjects exhibiting an antibody titer which is above a predetermined threshold. The skilled person knows that a threshold is defined for each vaccine and corresponding antigen. In the example using a hepatitis B virus vaccine, the threshold was set to 10 IU/ml. Using this threshold, there were two responders using the combination of the invention whereas no responders were detected in the placebo control or in the subjects treated with the same vaccine and a short chain inulin.

The effect observed on T cell response is preferably that:
the number of Th1 cells is increased by comparison to the number of Th1 cells in a control subject, preferably a subject having only received the vaccine or vaccine and short chain inulin (first aspect) (or in a subject having only received an antigen or an antigen and the long chain inulin, second aspect).

In the context of the invention, the word “increased” may mean an increase of at least 1%, 2%, 5%, 7%, 9%, 10%, 15%, 20%, 25%, 30%, 35%, 40%, 45%, 50%, 55%, 60%, 65%, 70%, 75%, 80%, 85%, 90%, 95%, 100%, 150%, 200% or more.

The assessment of the number of Th1 cells may be carried out on a subject, or in a sample from said subject such as in peripheral blood plasma samples. The technique used for the assessment of such titer may be FACS. The assessment of the number of such cells may be carried out as in the examples using TBET as marker of Th1 cells. In an embodiment, the assessment of the number of such Th1 cells is carried out after at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40 day of administration of the combination of the invention (i.e. first aspect, vaccine and long chain inulin) or of administration of an antigen and the long chain inulin (second aspect).

The increase of TNFalpha, IL-12 and/or IFNgamma production and the decrease of production of IL-10 and/or IL-6 by the long chain inulin is in line with the Th1 inducing profile or shifted Th1/Th2 balance of long chain inulin.

The preferred mode of administration for the long chain inulin in the first and second aspect of the invention is oral administration.

In an embodiment of the first aspect, the long chain inulin is orally administered before or after the vaccine has been administered. The long chain inulin may have been simultaneously or sequentially orally administered with the vaccine. It does not per se mean that the vaccine and the long chain inulin are formulated together in one composition. In an embodiment, the long chain inulin is orally administered before the vaccine is being administered. In this context, “before” may mean at least 10, 9, 8, 7, 6, 5, 4, 3, 2, 1 day before. In a more preferred embodiment, the long chain inulin is orally administered also after the vaccine has been administered. In this context, “after” means at least 1, 2, 3, 4, 5, 6, 7, 8, 9, 10 day after. It is therefore encompassed to orally administer the long chain inulin for a period such as 20 days and administer the vaccine once within this period. Depending on the vaccine in question, it may have to be administered once, twice, 3 times, 4 times or more. Accordingly the period of 20 days is not limited and may be extended for at least 10%, 30%, 50% 75%, 100% or more.

In an embodiment of the second aspect, the long chain inulin is orally administered on a daily basis during a given period of time. A period of time may be at least 1, 2, 3, 4, 5, 6 month or longer.

In the context of the invention, the word “composition” is used at least for two features of the invention. It is used in connection with the long chain inulin which may be formulated in a composition (first and second aspects). It is also used in connection with the term vaccine which may also be formulated in a composition (first aspect).

In a preferred embodiment, the long chain inulin (first and second aspects of the invention) used is formulated in a composition. Such composition is preferably suitable for oral administration. This composition is preferably a food composition such as a nutraceutical, a functional food, a food additive. More preferably said food composition is a food supplement, an infant food or follow-on formula or food for elderly people. Alternatively, this composition may be a medical food or a medical composition or considered as a medicament as defined below or later herein.

As used herein, “nutraceuticals” generally encompass foods or food products that provide health and medical benefits. Nutraceuticals are edible and may be eaten directly by humans, but are preferably provided to humans in the form of additives or nutritional supplements, e.g., in the form of tablets or capsules of the kind sold in health food stores, or as ingredients in edible solids, more preferably processed food products such as cereals, breads, tofu, cookies, ice cream, cakes, potato chips, pretzels, cheese, etc., and in drinkable liquids e.g., beverages such as milk, soda, sports drinks, and fruit juices. Especially preferred processes for producing nutraceuticals involve only naturally derived solvents.

Therefore, such a long chain inulin or composition comprising such a long chain inulin (first and second aspects of the invention) is preferably formulated as a food composition such as a nutraceutical, functional food, food supplement, food additive and is more preferably for use as a medicament. This medicament is preferably for preventing, treating, regressing, curing and/or delaying a disease or a condition associated with or caused by a pathogen.

In contrast to nutraceuticals, the so-called “medical foods” are not meant to be used by the general public and are not available in stores or supermarkets. Medical foods are not those foods included within a healthy diet to decrease the risk of disease, such as reduced-fat foods or low-sodium foods, nor are they weight loss products. A physician prescribes a medical food when a patient has special nutrient needs in order to manage a disease or health condition, and the patient is under the physician's ongoing care. The label states that the product is intended to be used to manage a specific medical disorder or condition. A long chain inulin as defined herein may therefore be comprised within a medical food composition.

Functional foods may encompass those foods included within a healthy diet to decrease the risk of disease, such as reduced-fat foods or low-sodium foods, or weight loss products.

It is also encompassed by the present invention that the long chain inulin is formulated in a composition with another carbohydrate. In an embodiment, another carbohydrate is a resistant starch (Bermudez-Brito M, et al, (2015), Mol. Nutr. Food Res., doi 10.1002/mnfr 201500148).

In an embodiment, a long chain inulin or a combination comprising said long chain inulin does not comprise a fucosyllactose. In said embodiment, preferably, a long chain inulin or a combination comprising said long chain inulin does neither comprise a fucosyllactose nor a betagalactooligosaccharide. In an embodiment, a long chain inulin is the sole or the only fructan used in the context of the invention. Preferably in this embodiment of said first and second aspect, there is no oligo fructose, more preferably no Raftilose P95.

In an embodiment, a vaccine or vaccine composition (first aspect) comprises an antigen as defined herein and optionally an adjuvant dissolved in PBS or a suitable buffer. Such composition may further comprise a pharmaceutically acceptable adjuvant and/or carrier. Such a composition is preferably for use as a medicine or as a medicament.

The vaccine or vaccine composition used in a combination of the invention may comprise an adjuvant. Any known adjuvant may be used herein. The skilled person knows several suitable adjuvants. Adjuvants are most preferably selected from the following list of adjuvants: cationic (antimicrobial) peptides, saponine and Toll-like receptor (TLR) ligands such as, but not limited to, poly(I:C), CpG motifs, LPS, lipid A, lipopeptide Pam3Cys and bacterial flagellins or parts thereof, and their derivatives having chemical modifications. Other preferred adjuvants for use in the method and in compositions according to the invention are: mixtures with live or killed BCG, immunoglobulin complexes with the said latency antigens or parts thereof, IC31 (from www.intercell.com; in WO03047602), QS21/MPL (US2003095974), DDA/MPL (WO2005004911), DA/TDB (WO2005004911; Holten-Andersen et al, 2004 Infect Immun. 2004 March; 72(3):1608-17.) and soluble LAG3 (CD223) (from www.Immunotep.com; US2002192195). In addition, another preferred adjuvant includes the use of Corynebacterium paryum or Propionobacterium acnes (Aebischer T., et al, (2000) Infection and Immunity., 68: 1328-1336, Poot J et al, (2009), Vaccine, 27: 4439-4446 and Ferreira J. H. et al, (2008), Vaccine, 26: 67-685).

A composition as defined in the first and second aspects of the present invention may be in the liquid, solid or semi-liquid or semi-solid form. A vaccine composition (first aspect) is preferably liquid. A long chain inulin composition (first or second aspect) is preferably liquid but may be solid or semi-solid.

In a preferred embodiment, other compounds are used sequentially or simultaneously with a combination of the invention (first aspect) or with a long chain inulin (second aspect) in order to improve the specificity of the treatment. It is advantageous for example to use other compounds that will further enhance the immune response of the treated subject. More preferably, such compounds are not present in a single composition together with the long chain inulin (first or second aspect) or together with the vaccine (first aspect).

A vaccine (or a vaccine composition) used in a combination of the invention (first aspect) may function as a therapeutic vaccine. Typically, there is a time period between contact with an antigen, i.e. infection and apparition of the first symptom of a disease associated with said antigen. In this case, a vaccine would act as a pharmacological immune product that would prevent and/or treat the disease and/or delay its progression by eliciting in the host an immune response that counteracts the pathological effect of the disease. A therapeutic vaccine differs from a prophylactic vaccine in that a therapeutic vaccine will induce protection in a subject who already has the infection or the disease. In another embodiment, a vaccine used in a combination of the invention is a prophylactic vaccine. A prophylactic vaccine may be administered to a subject before said subject has been contacted with said antigen.

A vaccine (or a vaccine composition) is preferably a medicament. In one embodiment, the long chain inulin composition used in a combination of the invention may also be considered as a medicament. A medicament as defined herein is preferably administered parenterally, e.g. by injection or infusion by intravenous, subcutaneous, intraperitoneal, intramuscular, intraarterial or intralesional route. A preferred administration mode for the vaccine is injection. The invention is not limited to a specific mode of administration of the vaccine as defined herein. A medicament may be combined with a pharmaceutically acceptable medium or delivery vehicle by conventional techniques known in the art. For example, a medicament may be dissolved in Phosphate buffer saline (PBS). Methods for preparing parenterally administrable compositions are well known in the art and described in more detail in various sources, including, for example, Remington's Pharmaceutical Sciences, Ed. A R Gennaro, 20th edition, 2000, Williams & Wilkins, PA, USA.

Alternatively, a vaccine as defined herein may be locally administered via a catheter or a pump, or a suppository. Alternatively, a vaccine as defined herein may be topically administered. The formulation of a vaccine as defined herein or of a composition comprising said vaccine depends on the intended mode of administration and (therapeutic) application. A pharmaceutical carrier can be any compatible, non toxic substance suitable to deliver said compound to a subject. E.g. sterile water, or inert solids or excipients may be used as the carrier, usually complemented with pharmaceutically acceptable adjuvants, buffering agents, dispersing agents, and the like. Compositions will either be in liquid, e.g. a stabilized suspension of said compound, or a composition comprising said compound, or in solid and/or dry forms: e.g. powder. For oral and rectal administration, said compound can be administered in solid dosage forms, such as capsules, tablets, suppositories, and powders, or in liquid dosage forms, such as elixirs, syrups, creams, ointments, enemas, and suspensions. Another form may be a semi-solid or semi-liquid form wherein said compound is present as a liquid form in, or on, a solid support such as a patch.

In a preferred embodiment of the first aspect, a combination is encompassed wherein the long chain inulin is orally administered and the vaccine is injected.

In a preferred embodiment of the second aspect, the long chain inulin is orally administered.

Accordingly a combination of the invention (first aspect) or a long chain inulin (second aspect) is preferably for use as a medicament. Such a medicament is preferably for preventing, treating, regressing, curing and/or delaying a disease or a condition associated with the antigen preferably present in the vaccine. The antigen is preferably from a pathogen as earlier defined herein.

In a preferred embodiment, such a combination (first aspect, more preferably the long chain inulin present in said combination) or such a long chain inulin (second aspect) influences the immune response against said antigen preferably present in a vaccine. The antigen is preferably from a pathogen as earlier defined herein.

More preferably, said influence is as earlier defined herein: cytokine production, B cell response, monocyte/macrophage response and/or T cell response.

In an embodiment, such influence may increase the ability of the human or animal immune system to fight such a disease or condition as defined herein.

Preferably, a therapeutically effective dose of a combination of the invention (first aspect, more preferably the long chain inulin present in said combination) or such a long chain inulin (second aspect) will prevent and/or delay the development of said disease or condition and/or it is able to elicit the proper immune response, or induce or induce an increase of the proper immune response against a specific antigen preferably present in the vaccine in a treated subject as defined herein. Even more preferably, the elicited or induced immune response is a protective immune response. Depending on the disease or the condition, the skilled person will know which parameter or symptom associated with the development of said disease to choose in order to follow the development of the disease or condition. Preferred parameters have already been defined herein to assess the effect of the long chain inulin preferably used in a combination of the invention on the immune system (i.e. B cell response, T cell response, monocyte/macrophage response and/or cytokine production).

In a more preferred embodiment, at least 5, 10, 15 or 20 micrograms of an antigen as defined herein is being used, preferably in a vaccine. Said vaccine may be administered at least once, twice, three times, four times or more. A vaccine, as defined herein, may be a prophylactic or a therapeutic vaccine. The volume in which an antigen as defined herein may be dissolved may vary from 100-500 microliters.

### Use

Accordingly, there is further provided the use of a combination as defined herein (first aspect) and/or corresponding compositions for the manufacture of a medicament for treating a disease or a condition associated with an antigen preferably as present in said vaccine as identified earlier herein.

Accordingly, there is further provided the use of a combination as identified herein (first aspect) and/or corresponding compositions for the manufacture of a medicament being a vaccine for treating a disease or a condition associated with the antigen present in said vaccine.

Accordingly, there is further provided the use of a long chain inulin or a composition comprising said long chain inulin as defined herein (second aspect) for the manufacture of a medicament for treating a disease or a condition associated with an antigen as identified earlier herein.

Each feature of these uses has already been defined herein. The antigen is preferably from a pathogen as earlier defined herein.

### Method of Treatment

In a further aspect, there is provided a method of treatment of a disease or a condition associated with an antigen as present in the vaccine, wherein said treatment comprises a combination (first aspect) and/or corresponding compositions.

Accordingly, there is further provided a method of treatment of a disease or a condition associated with an antigen as present in the vaccine as identified herein, wherein said treatment comprises a combination (first aspect) and/or corresponding compositions as defined herein.

In a further aspect, there is provided a method of treatment of a disease or a condition associated with an antigen, wherein said treatment comprises a long chain inulin or a composition comprising said long chain inulin as defined herein (second aspect). Each feature of these methods has already been defined herein. The antigen is preferably from a pathogen as earlier defined herein.

### Definitions

Polypeptide

“Polypeptide” as used herein refers to any peptide, oligopeptide, polypeptide, gene product, expression product, or protein. A polypeptide is comprised of consecutive amino acids. The term “polypeptide” encompasses naturally occurring or synthetic molecules. An antigen may be a polypeptide or a peptide or corresponding gene product.

Nucleic Acid Construct, Expression Vector, Operably Linked, Expression, Control Sequences

A nucleic acid construct is defined as a nucleic acid molecule which is isolated from a naturally occurring gene or which has been modified to contain segments of nucleic acids which are combined or juxtaposed in a manner which would not otherwise exist in nature. A nucleic acid molecule is represented by a nucleotide sequence. Optionally, a nucleotide sequence present in a nucleic acid construct is operably linked to one or more control sequences, which direct the production or expression of said peptide or polypeptide in a cell or in a subject.

“Operably linked” is defined herein as a configuration in which a control sequence is appropriately placed at a position relative to the nucleotide sequence coding for an antigen as defined herein such that the control sequence directs the production/expression of said antigen in a cell and/or in a subject.

Expression will be understood to include any step involved in the production of the peptide or polypeptide including, but not limited to, transcription, post-transcriptional modification, translation, post-translational modification and secretion.

Control sequence is defined herein to include all components which are necessary or advantageous for the expression of an antigen. At a minimum, the control sequences include a promoter and transcriptional and translational stop signals. Optionally, a promoter represented by a nucleotide sequence present in a nucleic acid construct is operably linked to another nucleotide sequence encoding a peptide or polypeptide as identified herein

An expression vector may be any vector which can be conveniently subjected to recombinant DNA procedures and can bring about the expression of a nucleotide sequence encoding a polypeptide in a cell and/or in a subject. As used herein, the term “promoter” refers to a nucleic acid fragment that functions to control the transcription of one or more genes or nucleic acids, located upstream with respect to the direction of transcription of the transcription initiation site of the gene. It is related to the binding site identified by the presence of a binding site for DNA-dependent RNA polymerase, transcription initiation sites, and any other DNA sequences, including, but not limited to, transcription factor binding sites, repressor and activator protein binding sites, and any other sequences of nucleotides known to one skilled in the art to act directly or indirectly to regulate the amount of transcription from the promoter. Within the context of the invention, a promoter preferably ends at nucleotide −1 of the transcription start site (TSS).

In this document and in its claims, the verb “to comprise” and its conjugations is used in its non-limiting sense to mean that items following the word are included, but items not specifically mentioned are not excluded. In addition the verb “to consist” may be replaced by “to consist essentially of” meaning that a product or a composition or a vaccine composition or a long chain inulin or a long chain inulin composition or a combination as defined herein may comprise additional component(s) than the ones specifically identified; said additional component(s) not altering the unique characteristic of the invention.

In addition, reference to an element by the indefinite article “a” or “an” does not exclude the possibility that more than one of the elements is present, unless the context clearly requires that there be one and only one of the elements. The indefinite article “a” or “an” thus usually means “at least one”.

The term “about” or “approximately” as used herein when referring to a measurable value such as a parameter, an amount, a temporal duration, and the like, is meant to encompass variations of +/−10% or less, more preferably +/−5% or less, and still more preferably +/−1% or less of and from the specified value, insofar such variations are appropriate to perform in the disclosed invention. It is to be understood that the value to which the modifier “about” or “approximately” refers is itself also specifically, and preferably, disclosed.

Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention.

Within the description, different aspects of the invention are defined in more detail in the form of several embodiments. Each embodiment so defined may be combined with any other embodiment or embodiments unless clearly indicated to the contrary. In particular, any feature indicated as being preferred or advantageous may be combined with any other feature or features indicated as being preferred or advantageous.

All patent and literature references cited in the present specification are hereby incorporated by reference in their entirety.

The following examples are offered for illustrative purposes only, and are not intended to limit the scope of the present invention in any way.

### DESCRIPTION OF THE FIGURES

FIG. 1. Chemical structure of inulin. G: glucose; F: fructose; norm: number of fructose moieties; DP (Degree of Polymerization)=n+1, or m+2

FIG. 2. DP profiles of supplement A and supplement B.

FIG. 3. Schematic overview of the human vaccination study, experimental procedures in time.

FIG. 4. Gating strategies for peripheral blood B lymphocytes, peripheral blood NK and NKT lymphocytes and peripheral blood T lymphocyte populations.

FIG. 4.a. Gating strategies for peripheral blood B lymphocyte populations. Single cells are gated using FSC-H vs. FSC-A scatter plot (A). Single cells are gated to a FSC vs. SSC plot, and lymphocyte gate is set (B). Within the lymphocyte population, B cells (CD19⁺) are identified as a separate population (C). The CD21 isotype is used to set the gate margin on 1% positive cells (D) and this gate is copied to the CD21 stained sample (E, upper plus lower gate), identifying CD21⁺ cells. Within this population, the CD27 isotype is used to set the gate margin for 1% positive cells (F, upper gate) and the CD27⁻ population (F, lower gate) and this gate is copied to the CD27 stained sample (G). Within the CD27⁻ population (G), IgD isotype is used to set the gate margin on 1% positive cells (H) and this gate is copied to the IgD stained sample (I), identifying the naïve non-class switched B cells. Within the CD19⁺ population, CD27 isotype is used to set the gate margin on 1% positive cells (J) and this gate is copied to the CD27 stained sample (K), identifying CD27⁺ cells. Using the IgD isotype, the 1% positive gate and negative gate are set (L), and the negative gate is copied to the IgD stained sample (M) to identify the IgD⁻ class switched memory population. Within this population, isotypes are used to set 1% positive gates for IgG (N) and IgA (Q), which are copied to IgG and IgA stained samples to identify IgG⁺ and IgA⁺ class switched memory cells (panel O and R). The isotype gate set in panel L is copied to the IgD stained sample to identify CD19⁺ CD27⁻ IgD⁺ cells (P). Within this population, the IgM isotype is used to set a 1% positive gate (S), which is copied to the IgM stained sample to identify the IgM⁺ non-class switched memory B cells (T). Within the CD19⁺ population, the CD38 isotype is used to set a 1% positive gate (U), which is copied to the CD38 stained sample to identify CD38⁺ and CD38^{hi} cells (V). Within the CD38^{hi} population the IgM isotype is used to set a 1% positive gate (W), which is copied to the IgM stained sample to identify IgM^{int} cells (X*) and the IgM^{hi} transitional B cell population (X**). The CD21^{lo} population as set in panel E is copied to this IgM^{int} cell population in panel X, identifying the plasmablasts and plasma cells (Y).

FIG. 4.b. Gating strategies for peripheral blood NK and NKT lymphocyte populations. Single cells and lymphocytes are gated as described for B lymphocytes (A,B). Within the lymphocyte population, CD56⁺ CD3⁺ cells are gated (C, **), indicating the NKT cells. Within the lymphocyte population, the CD3⁻ population is gated (C,*), and plotted in a panel with CD56 on the x-axis and CD16 on the y-axis (D). In panel D, left, the cytokine producing cells are gated [CD56^{hi} CD16⁺], and the cytotoxic cells are also gated, panel D, right [CD56^{int} CD16^{hi}]. Within these populations, isotype for CD161 (E and I), and isotype for CD335 (G and K) were used to set 1% positive gates, which were copied to the CD161 stained samples (F and J) and to the CD335 stained samples (H and L) respectively.

FIG. 4.c. Gating strategies for peripheral blood T lymphocyte populations. Single cells and lymphocytes are gated as described for B lymphocytes (A, B). The T cell population within lymphocytes are gated in a CD3 vs. CD8 plot (C). CTLs are identified and gated based on expression of CD3 and CD8 (D, upper population). The CD45RO isotype is used to set the gate margin on 1% positive CTLs (E). This gate is copied to the CD45RO stained sample, now demonstrating the CD45RO⁺ population, and the upper cell cluster now indicates CD45RO^{hi} cells (F). Th cells are identified based on expression of CD3 and no expression of CD8 (D, lower population). Isotype controls are used to set the gate margins on 1% positive cells for CD45RO, TBET, and RoRγT (G, I, L). These gates are copied to the CD45RO-, TBET-, and RoRγT stained samples, now demonstrating the positive populations (H, J, M). A cross-gate is applied to identify FoxP3+ Th cells (*) and CD294+ Th2 cells (**) in panel K.

FIG. 5. Activation of TLR2 by DP10-60 inulin-type fructans and DP2-25 inulin-type fructans. HEK239-Blue reporter cells for hTLR2 were stimulated with inulin-type fructans (dose is indicated in μg/mL), and TLR2-mediated activation of NF-κB was plotted as fold-induction of control. Statistical differences as compared to control were determined with a Kruskal-Wallis test and Dunn's post test, and are indicated with * (p<0.05), n=5. HKLM, heat killed Listeria monocytogenes, 10⁸ cells/mL.

FIG. 6. Human PBMC cytokine profiles induced by in vitro stimulation with inulin-type fructans of DP10-60 and DP2-25. Relative cytokine production as compared to unstimulated controls were plotted for PBMCs treated with DP10-60 inulin-type fructans or DP2-25 inulin-type fructans (depicted by black bars and white bars respectively). Wilcoxon signed rank test was used to test for differences in cytokine induction between the different fructan formulations, indicated with * (p<0.05), n=6.

FIG. 7. Anti-HBsAg antibody titer development in inulin-type fructan and placebo supplemented individuals in time. Statistical significance levels were determined with a Friedman test and Dunn's post test for individual supplements to test for titer development compared to basal samples at T0 (FIG. 7 a-c), and Mann Whitney for differences between supplements (FIG. 7 d). Median and IQR of the anti-HBsAg titers are plotted in IU/mL. Supplements were dosed at 8 g/d for 14 consecutive days around vaccination (n=13, n=13, and n=14 respectively for the supplemental groups DP10-60, DP2-25, and placebo). P=0.1 indicates a statistical trend, * represents statistical difference with p<0.05, and ** represents statistical difference with p<0.01.

FIG. 8. B cells and B cell subsets as percentages of lymphocytes, or within subsets in time, plotted per supplement. A) DP10-60 fructans, B) DP2-25 fructans, C) placebo. A Friedman test and a Dunn's post test were used to analyze time effect per supplement. Median and IQR are plotted as percentage of the indicated cell populations, n=13, 13, and 14 for supplement DP10-60, DP2-25, and placebo respectively. * represents statistical difference with p<0.05, and ** represents statistical difference with p<0.01.

FIG. 9. Percentages of cytokine producing NK cells and cytotoxic NK cells, and percentages of CD161+ and CD335+ cells within these populations, and NKT cells within the lymphocyte population in time, plotted per supplement. A) DP10-60 fructans, B) DP2-25 fructans C) placebo. Repeated measures ANOVA and Tukey's post test or a Friedman test and a Dunn's post test were used to analyze time effect per supplement. Median and IQR are plotted as percentage of the indicated cell populations, n=13, 13, and 14 for supplement DP10-60, DP2-25, and placebo respectively. P=0.1 indicates a statistical trend, * represents statistical difference with p<0.05, ** represents statistical difference with p<0.01, and * * * represents statistical difference with p<0.001.

FIG. 10. T cell subsets expressed as percentages of Th cells or T memory cells in time, plotted per supplement. A) DP10-60 fructans, B) DP2-25 fructans C) placebo. A Friedman test and a Dunn's post test were used to analyze time effect per supplement. Median and IQR are plotted as percentage of the indicated cell populations, n=13, 13, and 14 for supplement DP10-60, DP2-25, and placebo respectively. * represents statistical difference with p<0.05.

FIG. 11. Model representing the effects of inulin-type fructan supplementation on gut microbiota, SCFA, and the innate and adaptive immune system in humans.

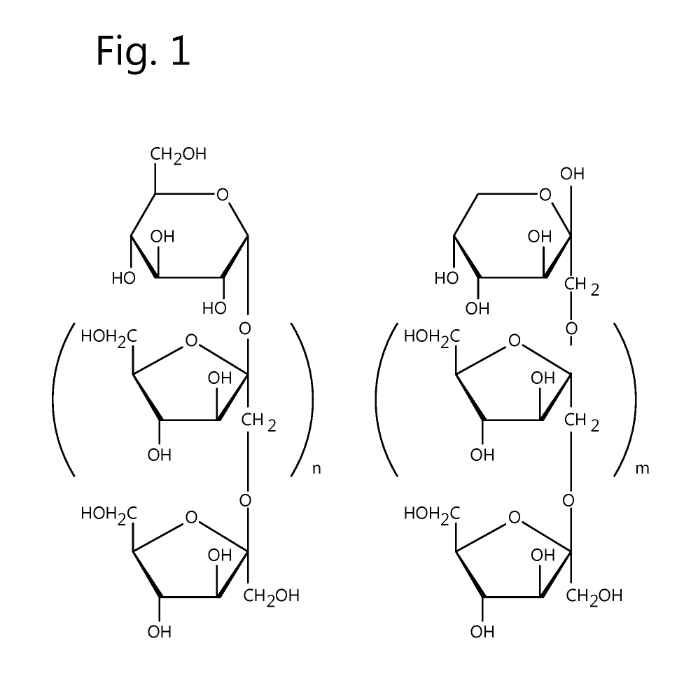

### EXAMPLES

### Methods

### Investigational Compounds

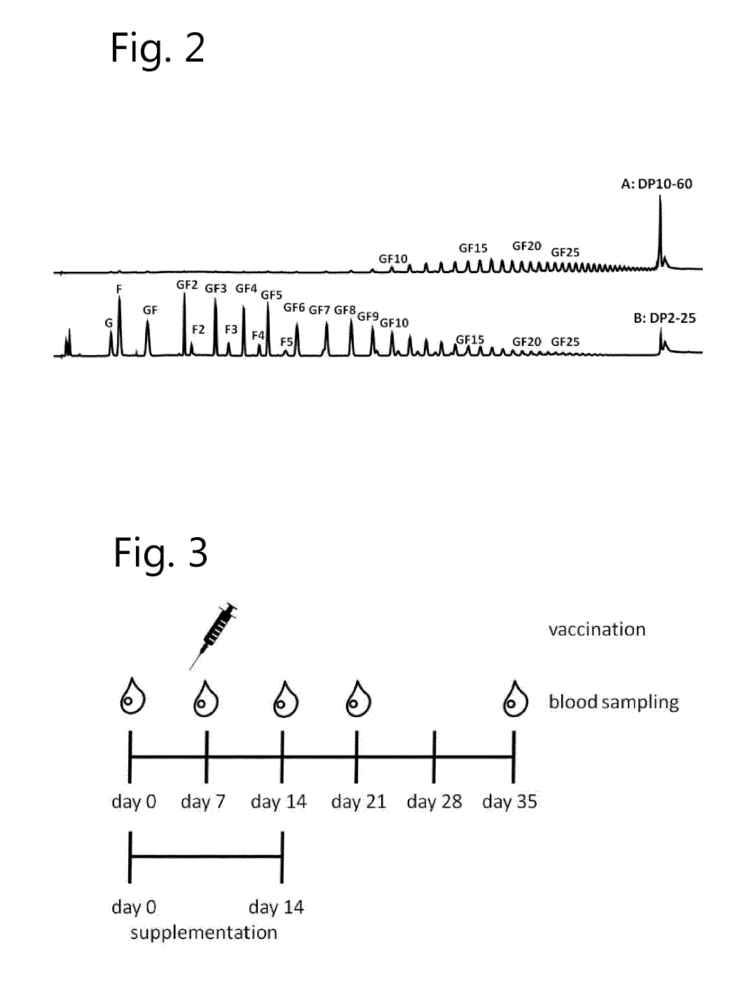

Inulin from chicory is a polydisperse mixture of linear fructan oligomers and polymers coupled by means of β(2-1) bonds, and mostly with a terminal glucose unit. The number of fructose units in the chain (degree of polymerization, or DP) can vary naturally between 2 and 60. Supplement A (Frutafit®TEX! Sensus, Roosendaal, the Netherlands), is a natural powdered food ingredient based on chicory inulin, and known for its texturizing properties. The DP ranges between mainly between 10 and 60 and average DP of approximately 14-30. Supplement B (Frutafit®CLR Sensus), is a powdered fructo-oligosaccharide (FOS) produced by partial hydrolysis of chicory inulin (Meyer & Blaauwhood, 2009). Frutafit®CLR is a highly soluble food ingredient with a DP ranging mainly between 2 and 35 but average DP of approximately 7-9. Supplement C consists of fructose, which is a powdered carbohydrate, and serves as placebo because it consists of the monomer building blocks of fructans and does not have β(2-1) bonds. The DP profiles of supplement A and B are depicted in FIG. 2.

### Volunteers and Interventions

This study was approved by the ethical board of the University Medical Center Groningen, Medisch Ethische Toetsingscommissie University Medical Center Groningen, and documented in the approved application METC_097. It has been registered in the national Dutch trial register, Nederlands Trial Register (NTR41644). Written informed consent was obtained from all participants, and data was analyzed and presented anonymously. Hepatitis B vaccination and blood sampling was conducted within the University Medical Center Groningen, in the Netherlands. All clinical investigation was conducted according to the principles expressed in the Declaration of Helsinki.

A randomized double-blind placebo controlled human dietary intervention trial was designed to study the effect of inulin-type fructans on vaccination efficacy and peripheral blood lymphocyte populations. To study whether the degree of polymerization (DP) of inulin-type fructans can influence whether different types of immune reactions are stimulated, three groups were included in the study. Healthy volunteers without a history of gastrointestinal symptoms and free of medication, aged 18-29 (17 males, 23 females), were supplemented for 14 days with inulin-type fructans of DP10-60, or with inulin-type fructans of DP2-25, or fructose (Sigma-Aldrich, the Netherlands) as placebo (n=13, 13, and 14 per group respectively, 8 g/d in one dose per day), and vaccinated against hepatitis B (Engerix-B, GlaxoSmithKline Biologicals s.a, Belgium) on day 7. The volunteers consumed their habitual diet and filled out a nutrition diary for the 35 days of the study. Blood samples were collected at day 0, 7, 14, 21, and 35, see FIG. 3 for a schematic overview of experimental interventions.

### Multiplex Cytokine Analysis of Human Peripheral Blood Mononuclear Cells

Human PBMCs of 6 healthy volunteers were isolated and cultured as previously described²⁰. 1×10⁶ cells were stimulated overnight with inulin-type fructans of DP10-60 or DP2-25 (100 μg/mL), and with plain culture medium as control. Cytokine profiles in the supernatant were assayed with a human Bio-PlexTM 6-plex premixed cytokine assay (group I, IL-1Ra, IL-1β, IL-6, IL-10, IL-12p70, and TNF-α) according to the manufacturer's instructions (Bio-Rad Laboratories, Veenendaal, The Netherlands), and analyzed as described previously²⁰.

### TLR2 Reporter Cell Culture and Reporter Assays

A HEK(293)-Blue reporter cell line with inserted constructs for human TLR2 and secreted embryonic alkaline phosphatase (SEAP), coupled to the NF-κB/AP-1 promotor, was purchased from Invivogen (Toulouse, France), and cultured as previously described²⁰. 2.8×10⁵ cells were stimulated overnight with a concentration series of DP10-60 and DP2-25 (100, 200, 400, 1000 and 2000 μg/mL), with plain culture medium as control, and with heat killed Listeria monocytogenes (1×10⁸ cells/mL, Invivogen). Reporter assay and analysis were performed according to the manufacturer's instructions, as previously described²⁰.

### Peripheral Blood Lymphocyte Isolation and Multi-Parameter Flow Cytometry

Multi-parameter flow cytometry was performed to measure percentages of different B cell, T cell, NK cell, and NKT cell populations within the total lymphocyte population and subsets within populations. Blood was drawn from the inner cubital vein and collected in 10 mL lithium heparin Vacutainer tubes (BD, Plymouth, UK). All subsequent steps were performed at 4° C. Whole blood (1.5 mL) was centrifuged at 2000 g for 15 min, and plasma supernatant was aliquotted and stored at −20° C. for anti-hepatitis B antibody titer analysis. The remaining whole blood was separated into two 50 mL tubes and erythrocytes were lyzed by incubating twice with 40 mL of ammonium chloride per tube for 10 min. Cell pellet was collected by centrifuging for 5 min at 1800 g. After washing the cell pellet twice with 15 mL FACS buffer (2% fetal bovine serum in phosphate buffered saline, FBS in PBS), cells were counted on a coulter counter (Beckton Dickinson, the Netherlands) and 1×10⁶ cells per well were transferred to a round bottom 96 wells plate. After pelleting the cells for 5 min at 1800 g and discarding the supernatant, cell pellets were resuspended in 50 μl of blocking buffer (20% normal rat serum, Jackson laboratories, in FACS buffer) and incubated for 20 min. Cells were pelleted as described above, resuspended in 50 μl of extracellular antibody mix consisting of extracellular antibodies, 5% normal rat serum, and FACS buffer (antibodies are listed in Table 1-3), and were incubated in the dark for 30 min. After washing the cells which were stained for B cell and NK cell markers, twice with 200 μl FACS buffer per well, cells were incubated with 200 μl of FACS-lysing buffer (Beckton Dickinson BV, Breda, the Netherlands) for 30 min. Cells were then washed three times with FACS buffer, resuspended in 200 μl FACS buffer per well, and stored at 4° C. in the dark until analysis. After incubation with the extracellular antibody mix, the cells stained for T cell markers were washed three times with 200 μl of permeabilization buffer (eBioscience, Vienna, Austria) per well. Cells were resuspended in 50 μl of intracellular blocking buffer (20% normal rat serum in permeabilization buffer) and incubated for 20 min. After pelleting the cells as described above and discarding the supernatant, cells were incubated with 50 μL of intracellular antibody mix, consisting of intracellular antibodies, 5% normal rat serum, and permeabilization buffer (antibodies are listed in Table 2), and incubated for 30 min. Cells were washed three times with permeabilization buffer, resuspended in 200 μl of FACS buffer, and stored at 4° C. in the dark until analysis on an LSR II flow cytometer (Beckton Dickinson BV). The corresponding isotype control antibodies were purchased from the same company as the target antibodies, and isotype stainings were used to set positive gates, using 1% margins. UltraComp eBeads (eBioscience, Vienna, Austria) were applied to set the appropriate compensation values for each antibody panel. FlowJo VX software (FlowJo, Oregon, USA) was used to analyze lymphocyte subsets. Basal sampling at day 0 for every individual allowed repeated measures analysis on the flow cytometry data.

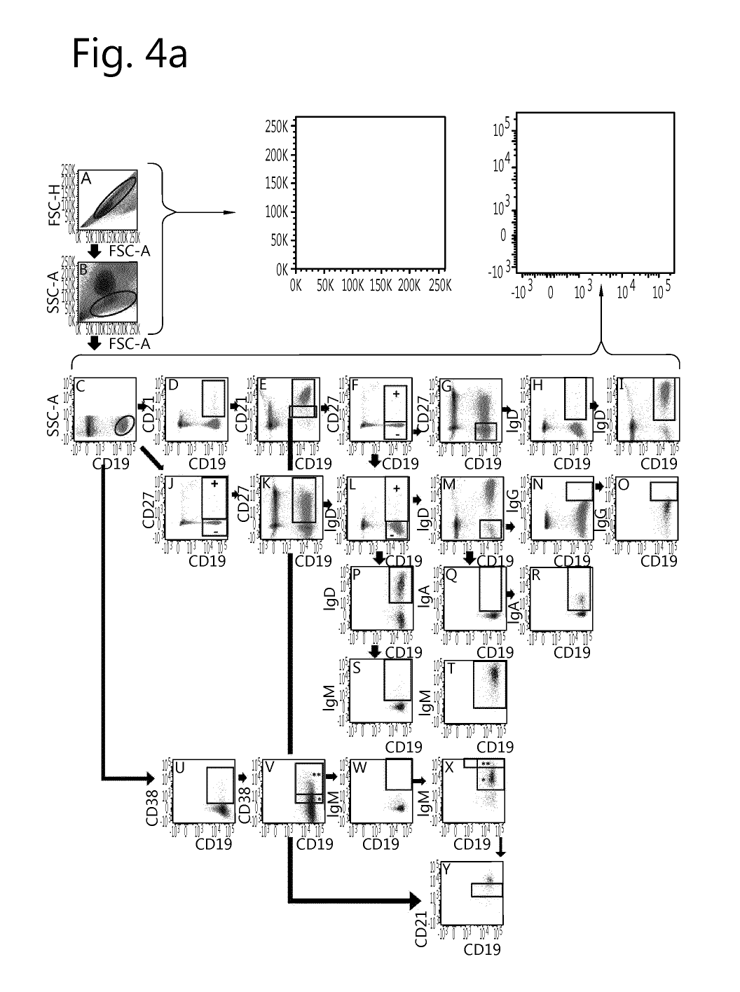

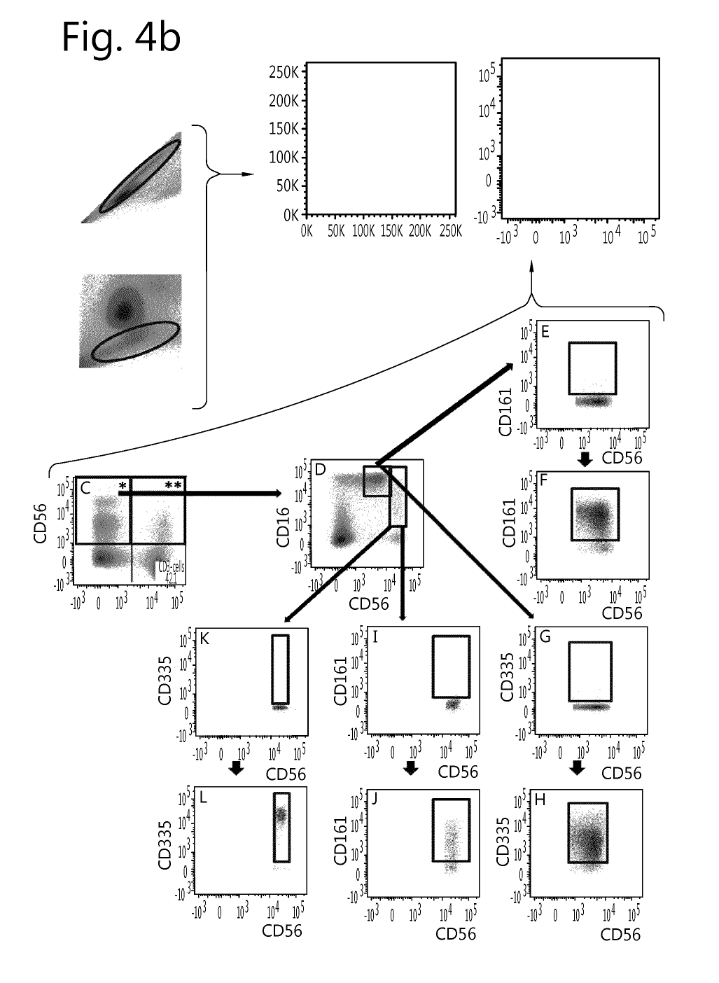

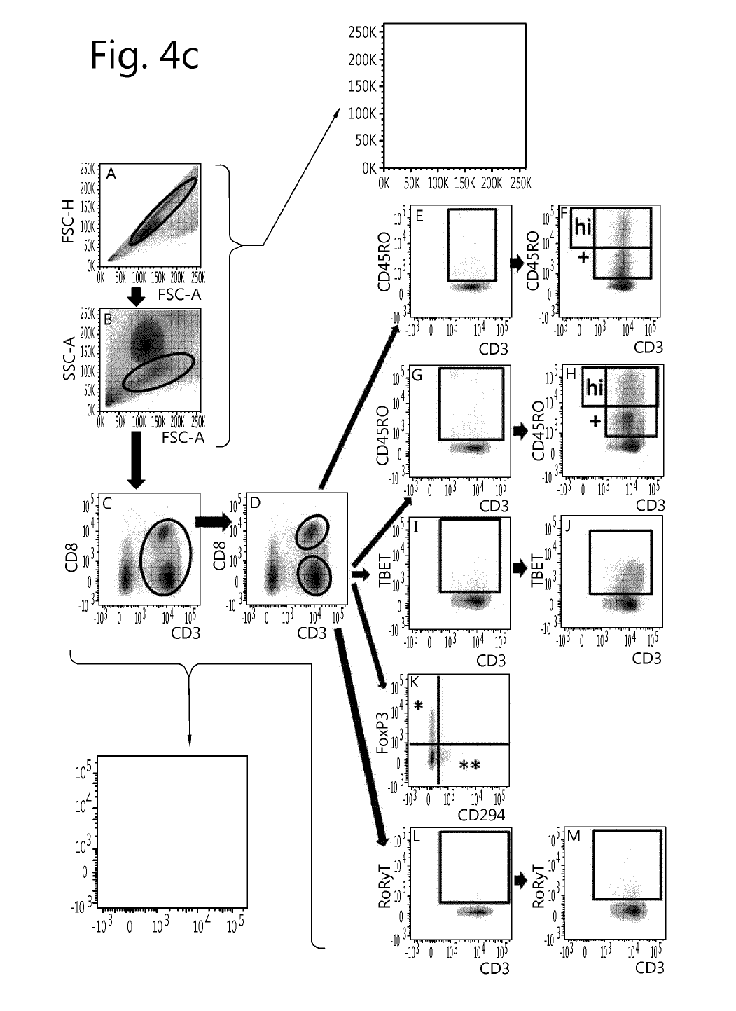

Flow cytometry gating strategies are described in FIG. 4. Within the B cell population we used the markers CD19, CD21, CD27, CD38, IgA, IgD, IgG, and IgM to identify naïve non-class switched B cells, class switched memory B cells (IgG⁺ and IgA⁺), non-class switched memory B cells, transitional B cells, and plasmablasts/plasma cells. Using CD3, CD56, and CD16 as markers we identified NK-, and NKT cells, and within the NK cell population we identified cytotoxic NK cells (CD56⁺,CD16^{hi})²⁷ and cytokine producing NK cells (CD56^{hi},CD16^{dim} or CD56^{hi},CD16⁻)²⁵. We determined the percentage of CD161⁺ and CD335⁺ cells within the cytotoxic-, and cytokine producing NK cells. CD161 is a cell surface marker which can be present on different subsets of NK cells^{25, 26}. Ligation of CD161 is known to cause activation of Pi3K, PkB, Akt and ERK pathways and is important in the regulation of NK and NKT cell function^{25, 26}. CD335 is a natural cytotoxicity-triggering receptor also known as PCR1 or NKp46^{26, 27}. We used CD3 and CD8 as markers to distinguish Th cells and CTLs, and within the Th cell population we identified memory cells (CD45RO⁺), Th1 cells (TBET⁺), Th2 cells (CD294⁺), Th17 cells (RoRγT⁺), and FoxP3⁺ Th cells which may represent regulatory cells. It is generally believed that for the time frame following challenge with a virus or a viral vaccine, human CD8⁺ memory cells are principally found within the CD45RO^{hi} population^{27, 28}, therefore we also gated and analyzed this population.

### Anti-HBsAg Titer Analysis

Anti-HBsAg titers were analyzed at day 0, 14, 21, and 35. Per sample, 500 μL of plasma was aliquotted in Architect tubes (Abbott, Ill., U.S.A.), and analyzed for Anti-HBsAg titers using an Architect Immunoassay Analyzer (Abbott Diagnostics) following the manufacturer's instructions.

### Statistical Analysis

GraphPad Prism 5.0 was used for statistical analysis of all data. Flow cytometry data sets were analyzed for normal distribution using a d'Agostino Pearson test. TLR2 activation data was analyzed with a Kruskal-Wallis test and Dunn's post test to determine significant differences compared to control. Data from multiplex cytokine assays was analyzed with a Wilcoxon signed rank test to identify significant differences between fructan treatments. To test for anti-HBsAg antibody titer development per supplement, statistical significance levels were determined with a Friedman test and Dunn's multiple comparison test, comparing T14, T21, and T35 to T0 (basal samples), and Mann Whitney test was used for differences between supplements. Repeated measures ANOVA and Tukey's multiple comparison test or a Friedman test and Dunn's multiple comparison test were used to analyse the flow cytometry data, comparing T14, T21, and T35 to T0 (basal samples). P-values <0.05 were considered statistically significant, and p=0.1 was considered representative for a statistical trend.

### Results

### DP10-60 and DP2-25 Inulin-Type Fructans Induce Dose-Dependent TLR2 Activation and DP Determines the Induced Cytokine Patterns in Human PBMCs

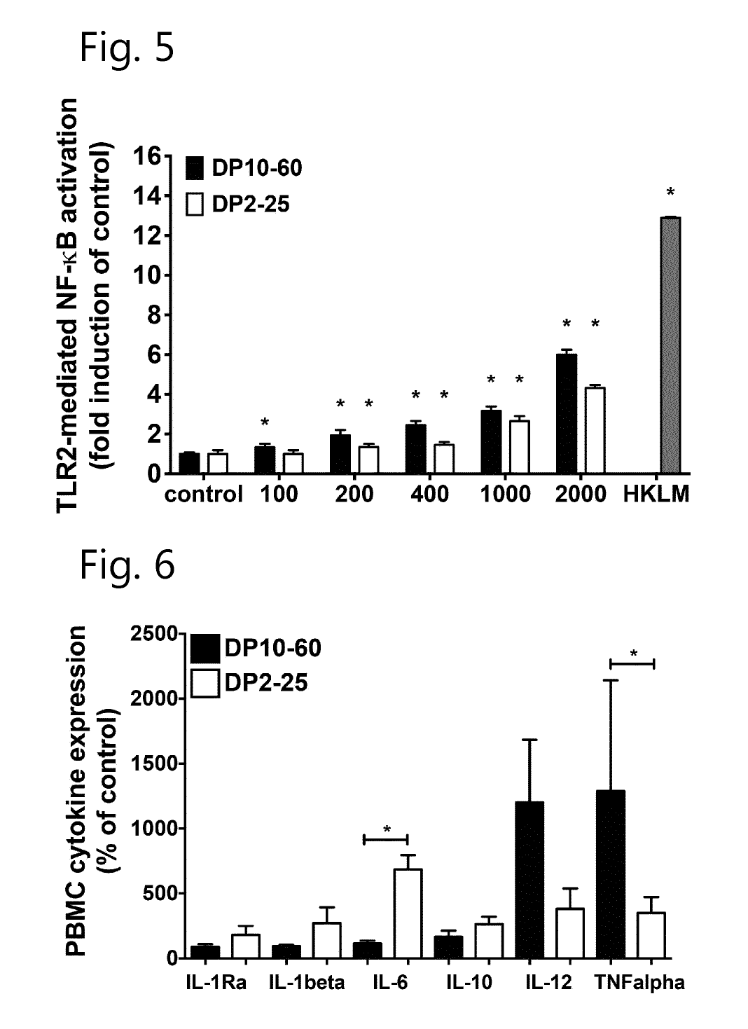

Upon 24 h of stimulation of HEK(293)-Blue hTLR2-reporter cells with DP10-60 or DP2-25 inulin-type fructans (FIG. 5), TLR2-mediated NF-κB/AP-1 activation was observed, which followed a dose-dependent pattern. TLR2 activation was not significantly different between DP10-60 and DP2-25 fructans. In human PBMCs (FIG. 6), TNFα production was induced by both fructan formulations, but was significantly increased by DP10-60 fructans as compared to DP2-25 fructans (1289.0±835.0% vs. 350.5±121.7%, p<0.05). IL-6 production was induced only by DP2-25 fructans but not DP10-60 fructans (685.0±109.9% vs. 114.7±21.3%, p<0.05). IL-12 production was induced by both formulations but did not differ between treatments.

Supplementation with DP10-60 Fructans Increased Anti-HBsAg Antibody Production Compared to Supplementation with DP2-25 Fructans and Placebo

Effects of nutritional supplementation with DP10-60 fructans vs. DP2-25 fructans on vaccination efficacy were studied by analyzing vaccine-induced anti-HBsAg antibodies in peripheral blood plasma samples of supplemented subjects using Architect Immunoassay analysis (FIG. 5). Subjects supplemented with DP10-60 fructans developed antibody titers from time point T21 (0.6±0.34 IQR) and reached significantly elevated levels at time point T35 compared to basal samples at T0 (2.93±0.75 IQR, p<0.05). In contrast, in plasma of subjects supplemented with DP2-25 fructans or placebo, no increases in antibody titers were observed at T35 as compared to basal samples at T0.

Individuals developing an antibody titer against any vaccination, which is above a predetermined threshold value are indicated as so-called responders, vs. individuals who do not reach titers above this value, which are indicated as non-responders. This threshold value for the anti-HBsAg applied in the present study is generally set at 10 IU/mL²⁹. There we no responders in the placebo groups and the DP2-25 fructan group at T35. This was different in the DP10-60 fructan group in which two responders were present at time point T35.

These results combined, demonstrate that supplementing young adults with a daily single bolus of 8 g DP10-60 fructans in the 14-day period around the first injection enhanced the efficacy of the anti-hepatitis B vaccination.

### Flow Cytometry Analysis of Peripheral Blood B Cell Subsets

Peripheral blood lymphocyte subsets of the supplemented individuals were analyzed by multi-parameter flow cytometry to study the effects of different DP fructans. Specifically, we were interested whether changes in T cell, B cell, NK cell, and NKT cell subsets were induced. As B cells are essential in mounting antibody responses against vaccines, we will first describe the results of B cell subsets in peripheral blood of subjects in the experimental groups. The subsets and their corresponding markers of identification are summarized in Table 1, gating strategies are shown in FIG. 4, and results of B lymphocyte flow cytometry are shown in FIG. 6. The percentage of B cells within the lymphocyte population was analyzed, and subsequently, the percentage of the following populations within the lymphocyte population or within the B cell population were analyzed and compared to basal samples at T0; 1) Naïve non-class switched B cells [CD19⁺ CD21⁺, CD27⁻ IgD⁺], 2) Class-switched memory B cells [CD19⁺ CD27⁺ IgD⁻; within this population the percentages of IgA⁺ and IgG⁺ cells were analyzed], 3) Non-class switched memory B cells [CD19⁺ CD27⁺ IgD⁺ IgM⁺], 4) transitional B cells [CD19⁺ CD38^{hi} IgM^{hi}], and 5) plasmablasts or plasma cells [CD19⁺ CD38^{hi} IgM^{int} CD21^{lo}].

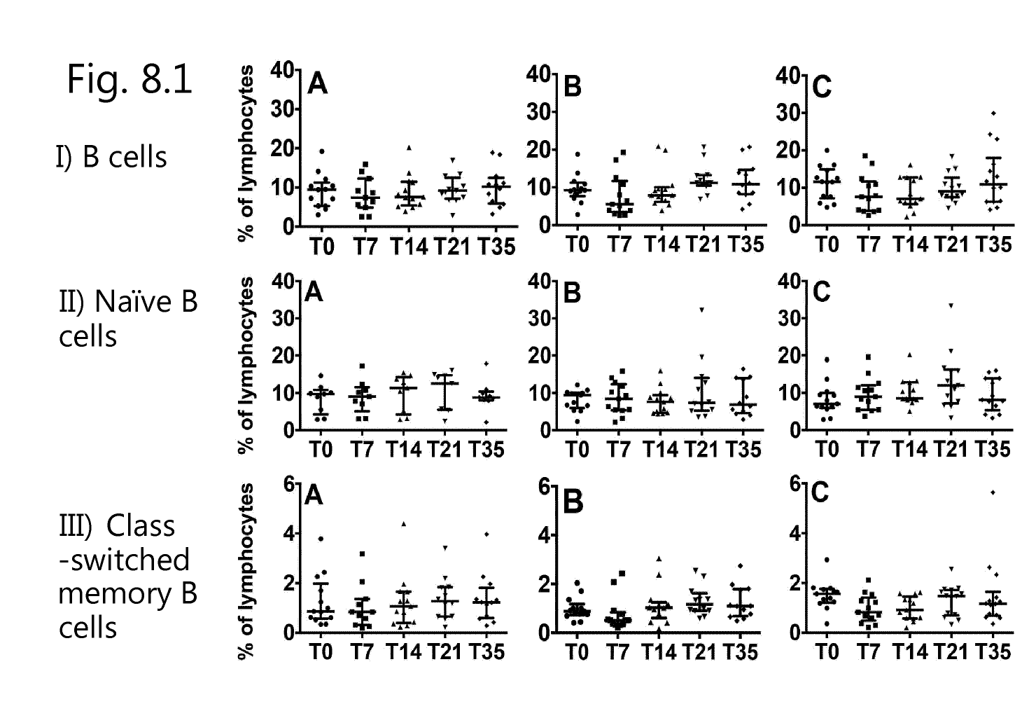

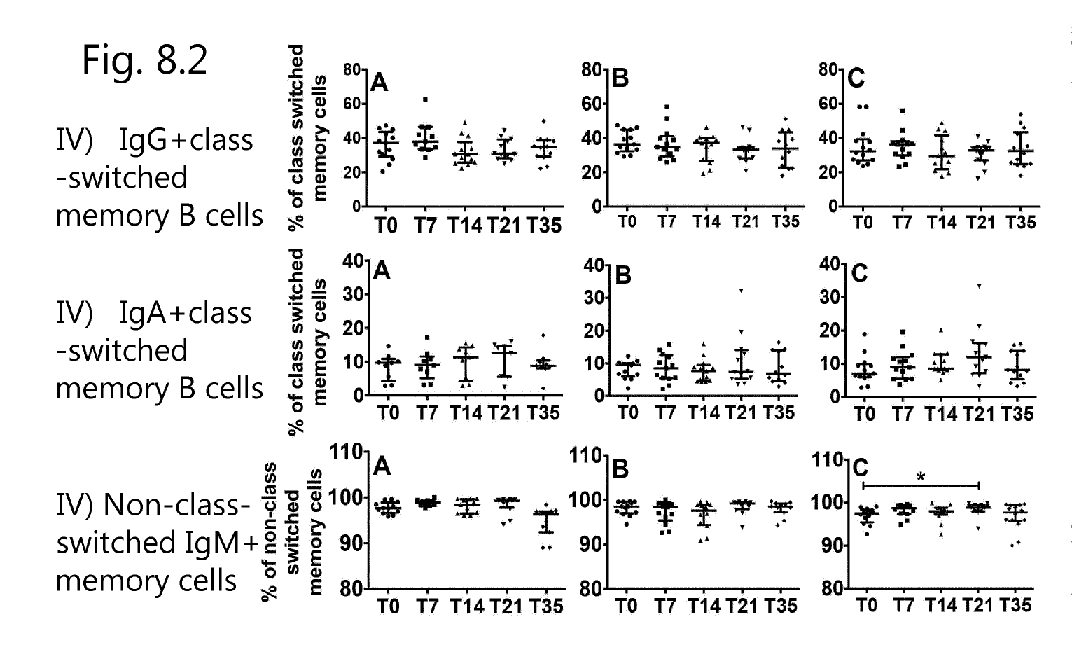

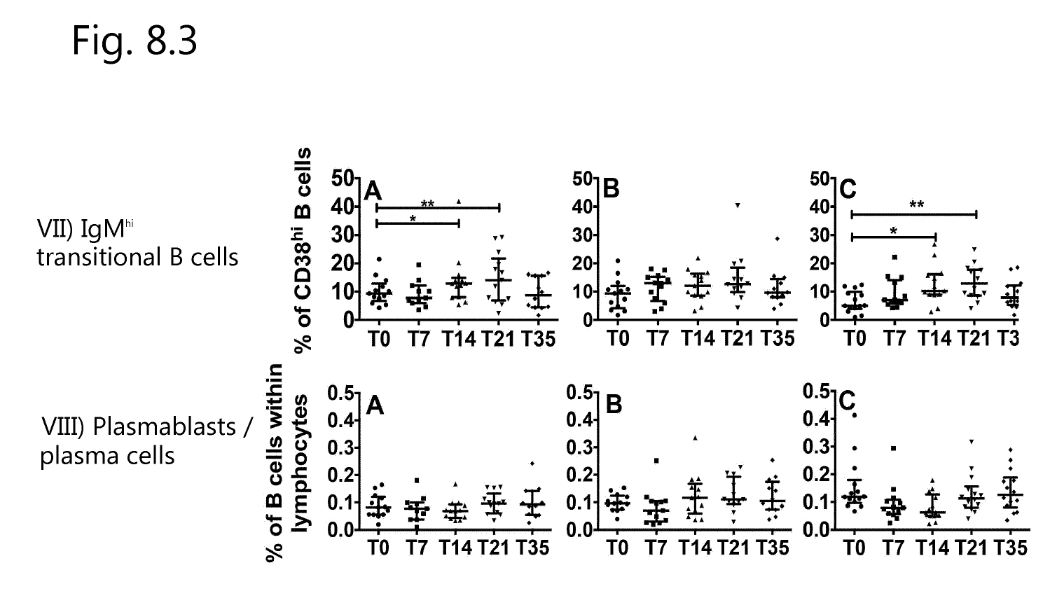

The percentages of B cells in the total lymphocyte population did not change as compared to basal samples (FIG. 8 part I). Naïve non-class switched B cells (FIG. 8 part II), class switched memory B cell populations (FIG. 8 part III), and non-class switched memory B cells (data not shown) did not differ in time compared to basal samples as percentage of lymphocytes or B cells. The percentage of IgM⁺ cells in the non-class switched memory B cell population (FIG. 8 part IV) was increased for the placebo group at T21 compared to the basal samples (p<0.05), but in the DP10-60 group and DP2-25 group no changes were observed as compared to basal samples. The percentage of transitional B cells and the percentages of plasmablasts or plasma cells did not differ in time as percentage of lymphocytes or B cells (FIG. 8 part V and 8 part VII). However, in the DP10-60 fructan group and the placebo group, the percentages of IgM^{hi} cells within the CD38^{hi} transitional B cells were significantly increased at T14 and T21 as compared to basal samples (p<0.05). This effect was absent in the DP2-25 fructan group (FIG. 8 part VI).

### Flow Cytometry Analysis of Peripheral Blood NK-, and NKT Cell Subsets

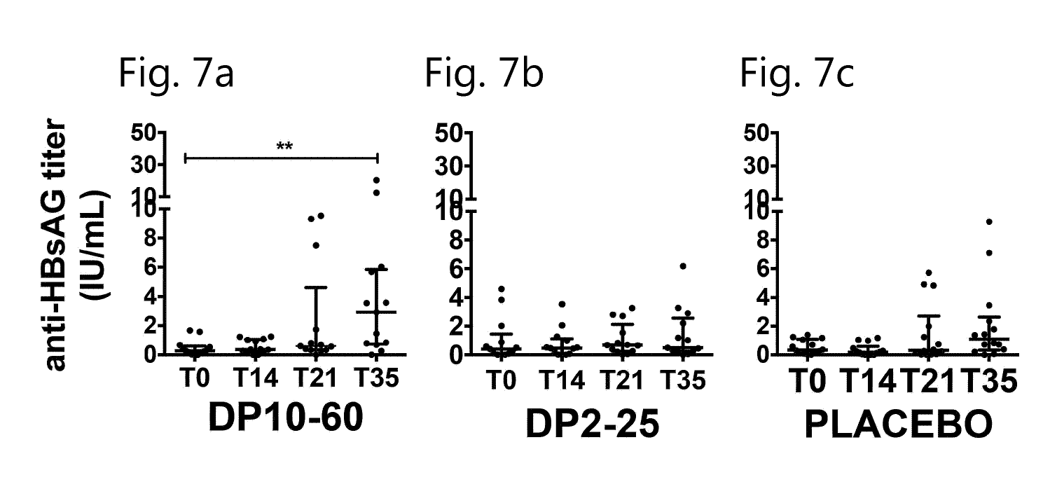

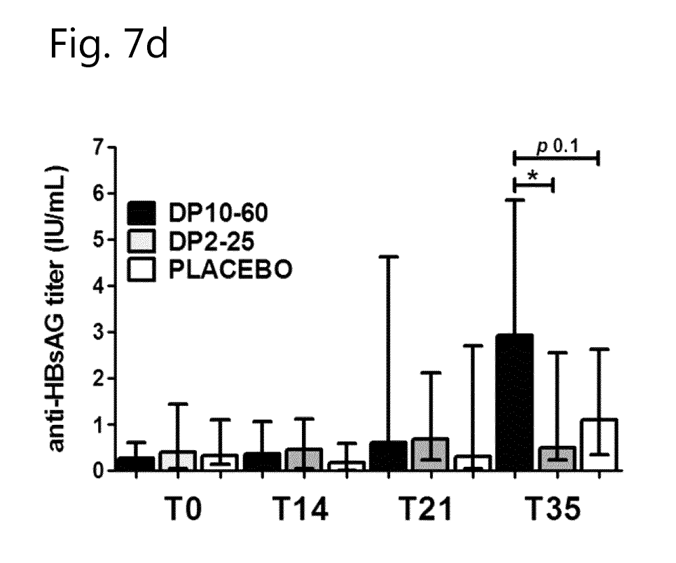

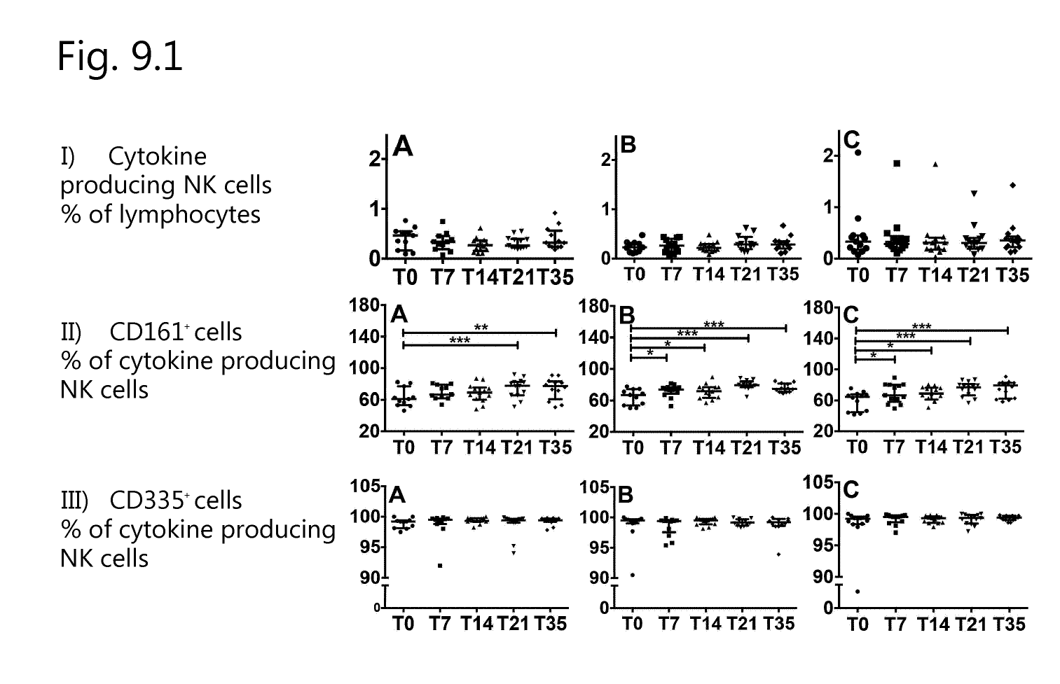

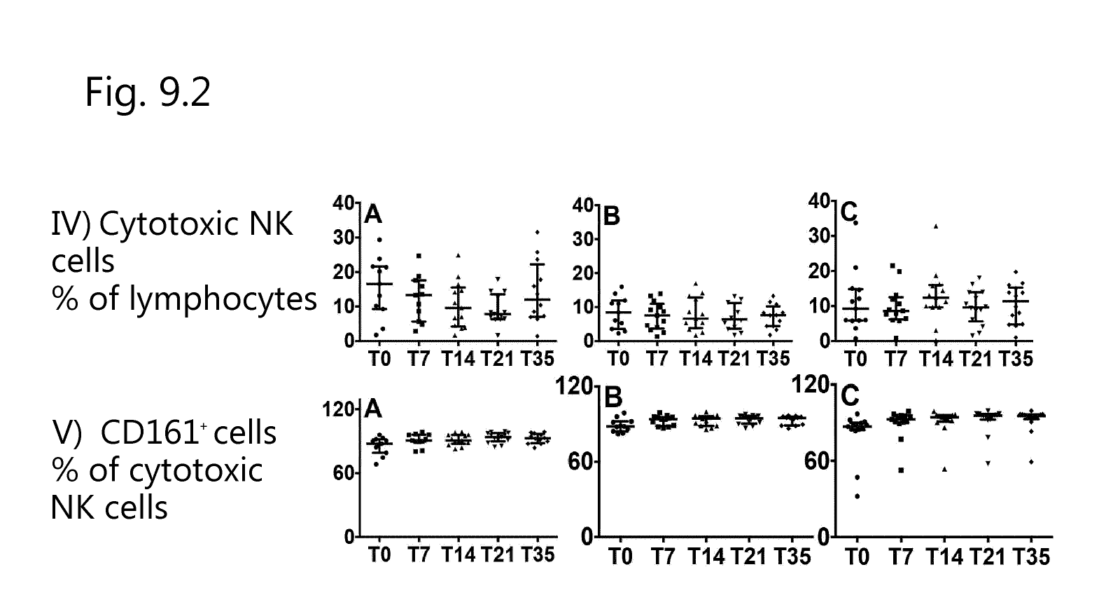

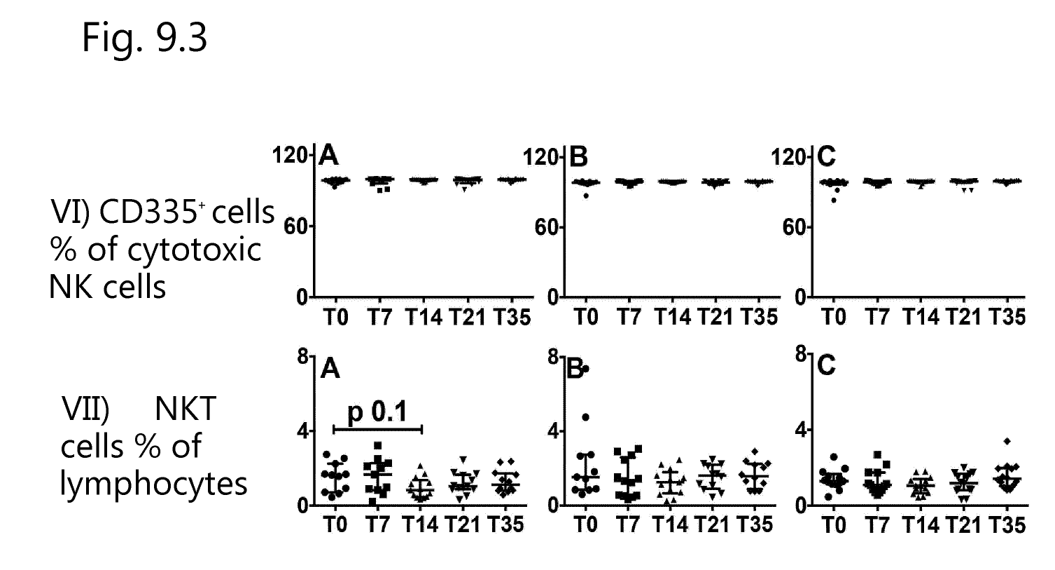

Human NK cells (CD56⁺ CD3⁻) are part of the first line of defense against viral pathogens and their activation can modulate the outcome of the adaptive immune response³⁰. Similar to NK cells, NKT cells (CD56⁺ CD3⁺) are also implicated in the response against hepatitis B vaccine antigens³⁰. The subsets and their corresponding markers of identification are summarized in Table 2, gating strategies are depicted in FIG. 4, and results of NK lymphocyte flow cytometry are depicted in FIG. 7. Within the lymphocyte population and within the NK cell population, the percentages of cytokine producing (CD56^{hi} CD16^{int}) or cytotoxic (CD56^{int} CD16^{hi}) cells were determined (FIG. 9 parts I and IV), and within the cytotoxic and cytokine producing cells, the percentages of CD161⁺ and CD335⁺ cells were analyzed. No differences were observed for either cytotoxic or cytokine producing NK cell populations as percentage of the lymphocyte population. In the DP2-25 group and placebo group (FIG. 9 part II), an increased effect was observed for CD161⁺ cytokine producing NK cells at all time points compared to basal samples (T0) but in the DP10-60 group this effect was only observed on time points T21 and T35 compared to basal samples (p<0.05). No changes were observed for CD161⁺ cytotoxic NK cells as compared to basal samples irrespective of the treatment. The percentage of CD335⁺ cells did not differ in time within either NK cell population. In the DP10-60 group (FIG. 9 part VII), NKT cells tended to show a decrease on time point T14 compared to basal samples, expressed as percentage of the lymphocyte population (p=0.1), but no changes were observed in the DP2-25 and placebo group.

### Flow Cytometry Analysis of Peripheral Blood T Cell Subsets

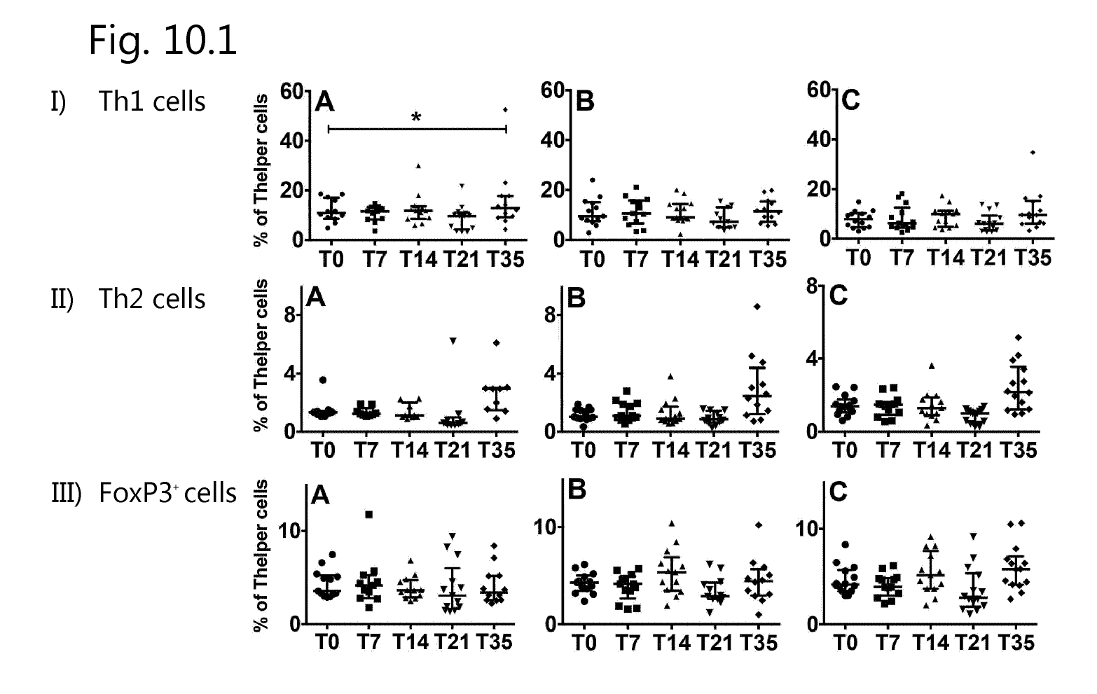

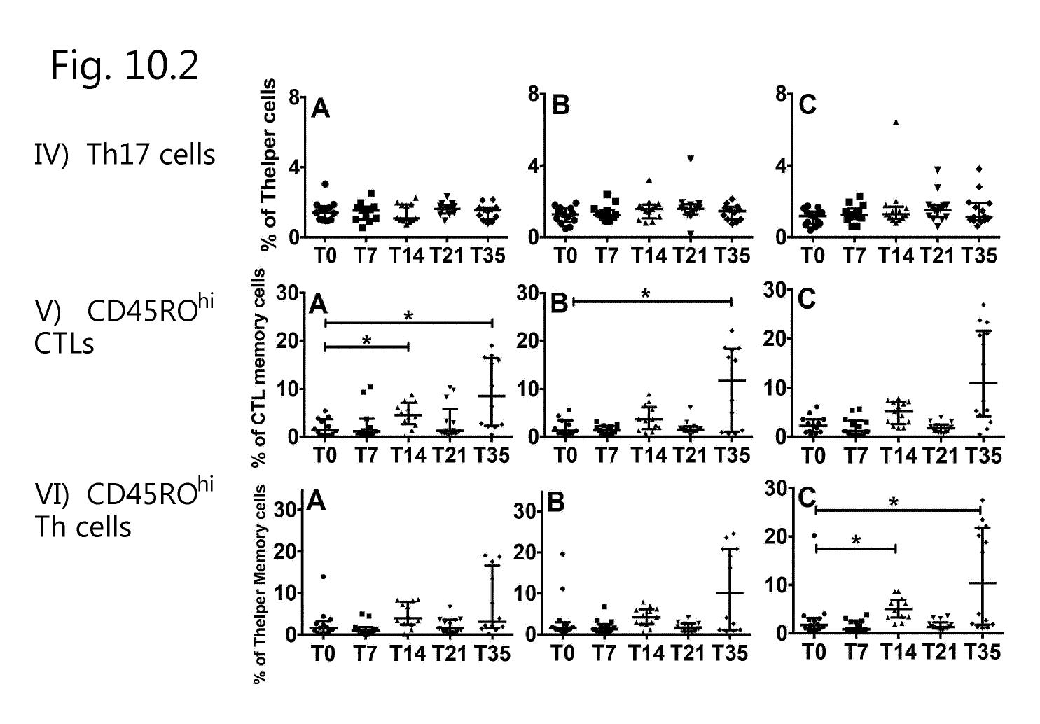

Gating strategies are depicted in FIG. 4, and T cell antibodies and labels are listed in Table 4. Results of T lymphocyte flow cytometry are shown in FIG. 7. Within the T cell population, we analyzed the percentages of Th1 cells, Th2 cells, FoxP3⁺ Th cells, Th17 cells, Th memory cells, and CTL memory cells. Percentages of Th2 cells, FoxP3⁺ Th cells, and Th17 cells at T35 did not differ from basal samples at T0 (FIG. 10 parts II, III, and IV). At time point T35, the percentage of TBET⁺ (Th1) cells in the DP10-60 fructan group was significantly increased as compared to basal levels (p<0.05), but this effect was not observed in the DP2-25 group or placebo group (FIG. 10 part I). Compared to basal samples at T0, the percentage of CD45RO^{hi} CTLs expressed as percentage of CTL memory cells was increased at time point T14 and T35 in the DP10-60 group (p<0.05), and at time point T35 in the DP2-25 group (p<0.05), but no significant increases were observed in the placebo group (FIG. 10 part V). In addition to the CTLs, Th cells also typically demonstrated changes in the CD45RO^{hi} population. However, only the placebo group demonstrated significant increases in CD45RO^{hi} Th cells as percentages of the Th memory population (FIG. 10 part VI), at time point T14 and T35 as compared to basal samples at T0 (p<0.05).

### Discussion

Previous studies from others and us have shown that inulin-type fructans can impact immunity by either serving as microbiota accessible fiber¹⁷ or by directly binding to immune cells²⁰, but scientific evidence in humans of related immunological benefits were largely lacking. To confirm the direct signaling mechanisms of inulin-type fructans on immune cells and to form an idea of the type of reactions of the immune system which could be expected to occur in vivo, we analyzed the TLR2-activating potential of the fibers and we studied whether DP10-60 induced different cytokines in human PBMCs compared to DP2-25. Both fibers dose-dependently activated TLR2 as expected, and the activation of NF-κB/AP1 through TLR2 appeared to be slightly stronger for DP10-60 fructans. Although the activation of NF-κB/AP1 via TLR2 can give an indication of the expected immune receptor ligation, analysis of the cytokines which are produced upon contact with the fibers by immune cells may give a more detailed view of the type of reactions which can be expected. The cytokine profile induced in PBMCs differed between the two formulations, indicating a more immunostimulating, Th1-inducing profile for DP10-60 fructans as measured by significantly increased TNFα production compared to DP2-25 fructans, together with induction of IL-12. DP2-25 fructans also induced IL-12 production, but this was accompanied by increased production IL-10 as compared to control, which is generally considered an immunosuppressive cytokine, and DP2-25 fructans also increased the production of IL-6 as compared to control and DP10-60 fructans, which has been shown to exert Th1-inhibiting properties³¹.

Building on the in vitro results in this study, we hypothesized that DP10-60 fructans would stimulate vaccine responses in vivo due to their predominant induction of proinflammatory cytokines in PBMCs, and the relatively low IL-10/IL-12 ratio²⁰, in combination with a strong ability to activate TLR2²⁰. We observed that supplementation with DP10-60 fructans significantly enhanced the titer response (T35 of the study) as compared to the DP2-25 fructan supplemented group, and that a strong increased trend was present as compared to the titer development in the placebo group. Another observation that supports the immune stimulating effects was the identification of two responders in the DP10-60 group vs. no responders in either the DP2-25 group or the placebo group. Inulin-type fructans have been studied in several infant vaccination trials, but in the majority the fructans were only studied in combination with GOS and/or pectic oligosaccharides. In one study though, long term supplementation with a mixture of oligofructose/inulin, i.e. short chain inulin-type fructans combined with long chain inulin-type fructans, enhanced the vaccination responses. In this study Saavedra et al.²¹ observed an increase in blood IgG levels after measles vaccination in a 10 week supplementation study with oligofructose (OF)/inulin (7/3, 0.2 g/kg BW/d) in 7-9 months old infants. In a study by Duggan et al.²⁴ in which 6-12 month old infants were supplemented with OF, i.e. only short chain inulin-type fructans (0.7 g/d), no effect was observed on antibody response after vaccination with H. influenza type B vaccine. Strikingly, other studies in infants with prebiotic mixtures did not induce vaccine potentiating effects^{22, 32}. It should be noted that the applied fructans in these mixtures are often—if not always—of a short chain nature. Dietary fibers can differ substantially in their molecular composition, and even within categories such as inulin-type fructans, different effects on the body and microbiota can be elicited¹⁵⁻¹⁹. To study the effect of different DP of fructans on changes in peripheral lymphocytes, we analyzed B cell-, T cell-, NK cell, and NKT cell subsets of supplemented individuals for their percentage of the total lymphocyte population, percentage of relevant subpopulations, and for differences in activation marker expression. Class-switching is one of the hallmarks of activation and maturation of B cells. If the peripheral blood is representative for the immune responses occurring after the vaccination, the increased titer response at T35 would be expected to coincide with a decrease in naïve B cells, and increases in transitional B cells or even plasma cells³³. The percentage of transitional B cells which are in the process of maturation (CD38^{hi} IgM^{hi}) was significantly increased in the DP10-60 group and the placebo group for time points T14 and T21 compared to the basal samples but not in the DP2-25 group. Increased percentages of this population indicate that B cells are activated and stimulated to differentiate into antibody producing plasma cells, which is a functional objective of vaccination. The fact that this population was stimulated by DP10-60 fructan supplementation and not by DP2-25 fructan supplementation underscores the effective differences of these two supplements. The induction of memory B cells is important for the ability to mount an efficient secondary immune response and protection against infection upon encountering the relevant antigen³³. In the non-class switched memory cells, the percentage of IgM⁺ cells was slightly increased for the placebo group at T21 compared to basal samples while the fructan groups did not. It is possible that the chosen time points for sampling are too early after vaccination to observe the induction of B memory cells³³ and that these cells arise after the 35 day period.

NK and NKT cells did not demonstrate clear supplement dependent effects, suggesting that B cells and especially T cells may be more involved in the boosting of the vaccination response via dietary inulin supplementation, or that B cell and T cell effects may be better detectable in peripheral blood. Supplementation with DP10-60 fructans induced striking differences in T cell populations in time, the increased titer response for the long chain group on T35 was associated with an increased percentage of (TBET⁺) Th1 cells compared to basal samples. Contrary to Th1 cells, (CD294⁺) Th2 cells did not change in time for either treatment. These results suggest that in the DP10-60 group, a shifted Th1/Th2 balance may have been induced which was skewed towards Th1 cell responses.

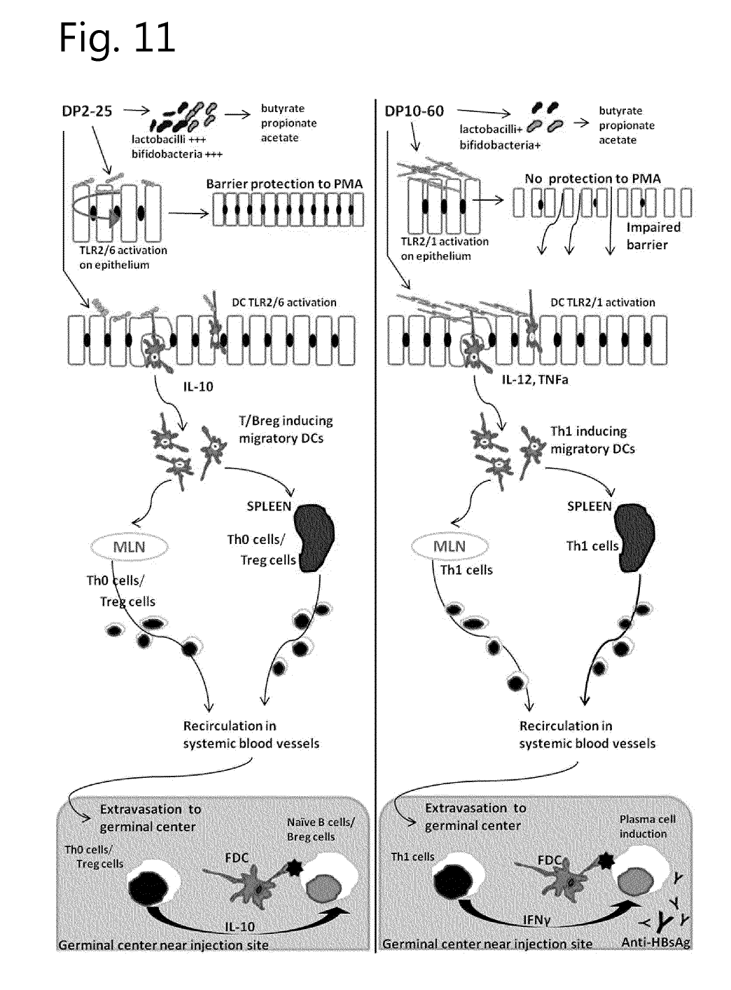

The fact that we observed clear differences for the different DP compounds suggests that DP is an important factor which determines the reaction of immune cells in the body. This could be explained by several mechanisms. Upon consumption, inulin-type fructans of different DP may selectively stimulate different populations of the microbiota^{34, 35, 36}, and these different populations could influence the immune system either in a stimulating or an attenuating manner³⁷. In addition, short chain fibers could be fermented into different products than long chain fibers, thus inducing different SCFA profiles in the intestine, qualitatively and quantitatively¹⁷. However, most effects of SCFA on the immune system are attenuating³⁸, contrary to the immune stimulation results observed in the current study. It is more likely that the effects are the result of the sum of indirect and direct effects on immune cells in the intestine^{17 19 20}. It is striking that an oral supplement can stimulate a systemic response to an intramuscular vaccination, and this warrants further studies into the way this process of antigen uptake and presentation can be impacted by orally taken supplements. Generally, the antigens of an intramuscular vaccination such as the applied hepatitis B vaccination, are thought to be detected by circulating dendritic cells, which then recruit other immune cells and migrate towards a draining lymph node, where antigen is presented to B-, and T cells followed by a primary immune response³⁹. Because of the natural surveillance function exerted by DCs, they circulate through the body and mount immune responses against antigens which are encountered. Ligation of innate immune receptors on DCs followed by antigen presentation toward effector cells, such as B cells, T cells, and NK cells locally, or in specialized lymphoid structures could be a mechanism which explains the induction of pro- and anti-inflammatory cytokines as observed in many prebiotic studies. Due to the fact that DCs can ‘sample’ the gut lumen⁴⁰ and they are migratory cells⁴¹, they are one of the candidate cell types to mediate dietary fiber-induced immune effects occurring in the periphery. A model of the impact of inulin-type fructans of different DP is depicted in FIG. 11.

In conclusion, this is the first in vivo study which demonstrates a structure-function relation of a dietary fiber on a systemic human immune response. We demonstrate that events in the gut which occur upon supplementing with a dietary fiber, have consequences for systemically induced changes in the immune system. The in vivo immunostimulatory potential of long-chain enriched inulin-type fructans subscribes an important nutritional health claim that these fibers can be beneficial for the immune system. Finally we feel that the use of a low efficacy vaccination model such as Hepatitis B may be more instrumental to demonstrate immunological effects of a nutritional supplement than the very efficient vaccination models^{21, 22, 32}. These human studies can be performed with relatively low numbers of subjects and still with a high statistical power.

TABLE 1
Antibodies applied for flow cytometry of B lymphocytes.

| Antibody and label | company | cat# | dilution |
|---|---|---|---|
| CD19-PE | ITK, Biolegend | 302208 | 10x |
| CD21-PE-Cy7 | ITK, Biolegend | 354912 | 100x |
| CD27-BV421 | ITK, Biolegend | 302824 | 50x |
| CD38-A700 | ITK, Biolegend | 303524 | 100x |
| IgA-FITC | DAKO | F0188 | 20x |
| IgD-APC | ITK, Biolegend | 348222 | 20x |
| IgG- PerCP-Cy5.5 | ITK, Biolegend | 409312 | 7.5x |
| IgM-BV605 | ITK, Biolegend | 314524 | 10x |

TABLE 2
Antibodies applied for flow cytometry of NK/NKT lymphocytes.

| Antibody and label | company | cat# | dilution |
|---|---|---|---|
| CD3-PerCP | ITK, Biolegend | 300428 | 30x |
| CD16-E450 | eBioscience | 48-0168-42 | 10x |
| CD56-APC | eBioscience | 17-0569-42 | 25x |
| CD335-PE | ITK, Biolegend | 331908 | 30x |
| CD161-PE-Cy7 | eBioscience | 25-1619-42 | 20x |

TABLE 3
Antibodies applied for flow cytometry of T lymphocytes.

| Antibody and label | company | cat# | dilution |
|---|---|---|---|
| CD3-Pacific Blue | BD | 558117 | 25x |
| CD8-PerCP | BD | 345774 | 25x |
| CD45RO-biotin | Biolegend | 304220 | 25x |
| Streptavidin-Pacific | LifeTechnologies | S32365 | 100x |
| Orange | | | |
| FoxP3-APC | eBioscience | 17-4776-42 | 25x |
| TBET-PE-Cy7 | eBioscience | 25-5825-80 | 150x |
| CD294-PE | Miltenyi | 130-098-879 | 40x |
| RoRγT-PE | eBioscience | 12-6981-80 | 50x |

### REFERENCE LIST

(1) Aune D, Chan D S, Lau R, et al. Dietary fibre, whole grains, and risk of colorectal cancer: systematic review and dose-response meta-analysis of prospective studies. BMJ (Clinical research ed.) 2011 Nov. 10; 343:d6617.
(2) Schulze M B, Schulz M, Heidemann C, et al. Fiber and magnesium intake and incidence of type 2 diabetes: a prospective study and meta-analysis. Archives of Internal Medicine 2007 May 14; 167(9):956-965.
(3) Pereira M A, O'Reilly E, Augustsson K, et al. Dietary fiber and risk of coronary heart disease: a pooled analysis of cohort studies. Archives of Internal Medicine 2004 Feb. 23; 164(4):370-376.
(4) Fardet A. New hypotheses for the health-protective mechanisms of whole-grain cereals: what is beyond fibre? Nutrition research reviews 2010 June; 23(1):65-134.
(5) Kaczmarczyk M M, Miller M J, Freund G G. The health benefits of dietary fiber: beyond the usual suspects of type 2 diabetes mellitus, cardiovascular disease and colon cancer. Metabolism: clinical and experimental 2012 August; 61(8):1058-1066.
(6) Vuksan V, Jenkins A L, Jenkins D J, et al. Using cereal to increase dietary fiber intake to the recommended level and the effect of fiber on bowel function in healthy persons consuming North American diets. The American Journal of Clinical Nutrition 2008 November; 88(5):1256-1262.
(7) Weber T K, Toporovski M S, Tahan S, et al. Dietary fiber mixture in pediatric patients with controlled chronic constipation. Journal of pediatric gastroenterology and nutrition 2014 March; 58(3):297-302.
(8) Kleessen B, Sykura B, Zunft H J, et al. Effects of inulin and lactose on fecal microflora, microbial activity, and bowel habit in elderly constipated persons. The American Journal of Clinical Nutrition 1997 May; 65(5):1397-1402.
(9) Marteau P, Jacobs H, Cazaubiel M, et al. Effects of chicory inulin in constipated elderly people: a double-blind controlled trial. International journal of food sciences and nutrition 2011 March; 62(2):164-170.
(10) Lopez Roman J, Martinez Gonzalvez A B, Luque A, et al. The effect of a fibre enriched dietary milk product in chronic primary idiopatic constipation. Nutricion hospitalaria: organo oficial de la Sociedad Espanola de Nutricion Parenteral y Enteral 2008 January-February; 23(1):12-19.
(11) Chuang S C, Norat T, Murphy N, et al. Fiber intake and total and cause-specific mortality in the European Prospective Investigation into Cancer and Nutrition cohort. The American Journal of Clinical Nutrition 2012 July; 96(1):164-174.
(12) Landberg R. Dietary fiber and mortality: convincing observations that call for mechanistic investigations. The American Journal of Clinical Nutrition 2012 July; 96(1):3-4.
(13) Gibson G R, Roberfroid M B. Dietary modulation of the human colonic microbiota: introducing the concept of prebiotics. The Journal of nutrition 1995 June; 125(6):1401-1412.
(14) Park Y, Subar A F, Hollenbeck A, et al. Dietary fiber intake and mortality in the NIH-AARP diet and health study. Archives of Internal Medicine 2011 Jun. 27; 171(12):1061-1068.
(15) Lomax A R, Calder P C. Probiotics, immune function, infection and inflammation: a review of the evidence from studies conducted in humans. Current pharmaceutical design 2009; 15(13):1428-1518.
(16) Kelly G. Inulin-type prebiotics: a review. (Part 2). Alternative Medicine Review: A Journal of Clinical Therapeutic 2009 March; 14(1):36-55.
(17) Roberfroid M, Gibson G R, Hoyles L, et al. Prebiotic effects: metabolic and health benefits. The British journal of nutrition 2010 August; 104 Suppl 2:S1-63.
(18) Rijnierse A, Jeurink P V, Garssen J, et al. Food-derived oligosaccharides exhibit pharmaceutical properties. European journal of pharmacology 2011 Jul. 28.
(19) Vogt L M, Meyer D, Pullens G, et al. Immunological properties of inulin-type fructans. Critical reviews in food science and nutrition 2013.
(20) Vogt L, Ramasamy U, Meyer D, et al. Immune modulation by different types of beta2-->1-fructans is toll-like receptor dependent. PloS one 2013 Jul. 5; 8(7):e68367.
(21) Saavedra J M, Tschernia A. Human studies with probiotics and prebiotics: clinical implications. The British journal of nutrition 2002 May; 87 Suppl 2:S241-6.
(22) Stam J, van Stuijvenberg M, Garssen J, et al. A mixture of three prebiotics does not affect vaccine specific antibody responses in healthy term infants in the first year of life. Vaccine 2011 Oct. 13; 29(44):7766-7772.
(23) Bunout D, Hirsch S, Pia de la Maza M, et al. Effects of prebiotics on the immune response to vaccination in the elderly. JPEN.Journal of parenteral and enteral nutrition 2002 November-December; 26(6):372-376.
(24) Duggan C, Penny M E, Hibberd P, et al. Oligofructose-supplemented infant cereal: 2 randomized, blinded, community-based trials in Peruvian infants. The American Journal of Clinical Nutrition 2003 April; 77(4):937-942.
(25) Nagler A, Lanier L L, Cwirla S, et al. Comparative studies of human FcRIII-positive and negative natural killer cells. Journal of immunology (Baltimore, Md.: 1950) 1989 Nov. 15; 143(10):3183-3191.
(26) Pozo D, Valés-Gómez M, Mavaddat N, et al. CD161 (Human NKR-P1A) Signaling in NK Cells Involves the Activation of Acid Sphingomyelinase. The Journal of Immunology 2006; 176(4):2397-2398-2406.
(27) Biassoni R, Cantoni C, Marras D, et al. Human natural killer cell receptors: insights into their molecular function and structure. Journal of Cellular and Molecular Medicine 2003 October-December; 7(4):376-387.
(28) Wills M R, Carmichael A J, Weekes M P, et al. Human virus-specific CD8+ CTL clones revert from CD45ROhigh to CD45RAhigh in vivo: CD45RAhighCD8+ T cells comprise both naive and memory cells. Journal of immunology (Baltimore, Md.: 1950) 1999 Jun. 15; 162(12):7080-7087.
(29) Hoebe C J, Vermeiren A P, Dukers-Muijrers N H. Revaccination with Fendrix® or HBVaxPro® results in better response rates than does revaccination with three doses of Engerix-B® in previous non-responders. Vaccine 2012 Nov. 6; 30(48):6734-6737.
(30) Albarran B, Goncalves L, Salmen S, et al. Profiles of NK, NKT cell activation and cytokine production following vaccination against hepatitis B. APMIS: Acta Pathologica, Microbiologica, et Immunologica Scandinavica 2005 July-August; 113(7-8):526-535.
(31) Diehl S, Rincon M. The two faces of IL-6 on Th1/Th2 differentiation. Molecular immunology 2002 December; 39(9):531-536.
(32) van den Berg J P, Westerbeek E A, van der Klis F R, et al. Neutral and acidic oligosaccharides supplementation does not increase the vaccine antibody response in preterm infants in a randomized clinical trial. PloS one 2013 Aug. 8; 8(8):e70904.
(33) Li J, Tan D, Liu H, et al. CD4(+) CD25(+) FoxP3(+) T regulatory cells in subjects responsive or unresponsive to hepatitis B vaccination. Zhong nan da xue xue bao. Yi xue ban=Journal of Central South University.Medical sciences 2011 November; 36(11):1046-1051.
(34) Alles M S, Hautvast J G, Nagengast F M, et al. Fate of fructo-oligosaccharides in the human intestine. The British journal of nutrition 1996 August; 76(2):211-221.
(35) Rumessen J J, Bode S, Hamberg O, et al. Fructans of Jerusalem artichokes: intestinal transport, absorption, fermentation, and influence on blood glucose, insulin, and C-peptide responses in healthy subjects. The American Journal of Clinical Nutrition 1990 October; 52(4):675-681.
(36) van de Wiele T, Boon N, Possemiers S, et al. Inulin-type fructans of longer degree of polymerization exert more pronounced in vitro prebiotic effects. Journal of applied microbiology 2007 February; 102(2):452-460.
(37) Smelt M J, de Haan B J, Bron P A, et al. Probiotics can generate FoxP3 T-cell responses in the small intestine and simultaneously inducing CD4 and CD8 T cell activation in the large intestine. PloS one 2013 Jul. 4; 8(7):e68952.
(38) Meijer K, de Vos P, Priebe M G. Butyrate and other short-chain fatty acids as modulators of immunity: what relevance for health? Current opinion in clinical nutrition and metabolic care 2010 November; 13(6):715-721.
(39) Siegrist C A. Vaccine Immunology. In: Plotkin S, Orenstein W, Offit P, editors. Vaccinese-book.: Elsevier; 2013; 17-18-36.
(40) Rescigno M, Urbano M, Valzasina B, et al. Dendritic cells express tight junction proteins and penetrate gut epithelial monolayers to sample bacteria. Nature immunology 2001 April; 2(4):361-367.
(41) Rivollier A, He J, Kole A, et al. Inflammation switches the differentiation program of Ly6Chi monocytes from antiinflammatory macrophages to inflammatory dendritic cells in the colon. The Journal of experimental medicine 2012 Jan. 16; 209(1):139-155.

1-17. (canceled)

18. A method for influencing the immune response against a pathogen, wherein a long chain inulin or a combination comprising a long chain inulin and a vaccine is orally administered.

19. The method according to claim 18, wherein the long chain inulin has a degree of polymeraizeation (DP) of 10-60.

20. The method according to claim 18, wherein the vaccine comprises an antigen from a pathogen.

21. The method according to claim 18, wherein the pathogen is a virus or a bacterium.

22. The method according to claim 21, wherein the virus is the hepatitis B virus.

23. The method according to claim 22, wherein the virus induces a systemic viral infection and/or the bacterium a systemic bacterial infection.

24. The method according to claim 18, wherein the long chain inulin is formulated in a composition.

25. The method according to claim 24, wherein the composition is a food composition.

26. The method according to claim 25, wherein the food composition is a food supplement, infant food or follow-on formula.

27. The method according to claim 18, wherein the dose of long chain inulin is ranged from:
1 to 25 g per day for human adult, or 1 to 8 g per day or 1 to 5 g per day,
1 to 15 g per day for children or small children, or 1 to 10 g per day or 1 to 8 g per day, or 1 to 5 g per day, or
0.01 to 15 g per day for baby, or 0.1 to 12 g per day or 0.5 to 8 g per day, or 1 to 5 g per day.

28. The method according to claim 18, wherein the long chain inulin is formulated with another carbohydrate.

29. The method according to claim 18, wherein the long chain inulin is orally administered before or after the vaccine has been administered.

30. The method according to claim 29, wherein the vaccine has been administered by injection.

31. The method according to claim 18, wherein the long chain inulin or the combination is a medicament.

32. The method according to claim 31, wherein the medicament is for preventing, treating, regressing, curing and/or delaying a disease or a condition associated with the antigen from the pathogen defined in claim 4.

33. The method according to claim 18, wherein oral administration of long chain inulin influences the immune response against an antigen present in a vaccine in at least one of the following ways:
cytokine production;
B cell response;
monocyte/macrophage response; and/or
T cell response.

34. The method according to claim 33, wherein the T cell response is such that the number of Th1 cells is increased by comparison to the number of Th1 cells in a control subject.

35. The method according to claim 18, wherein the combination or the long chain inulin is for adult or elderly.
